(19) United States
(12) Patent Application Publication
(10) Pub. No.: US 2009/0215709 A1
(43) Pub. Date: Aug. 27, 2009

(54) Methylation markers for diagnosis and treatment of cancers

(75) Inventors: Wim Van Criekinge, Sart-Tilman (Liege) (BE); Josef Straub, Sart-Tilman (Liege) (BE); David Sidransky, Baltimore, MD (US)

Correspondence Address: BANNER & WITCOFF, LTD., 1100 13th STREET, N.W., SUITE 1200, WASHINGTON, DC, 20005-4051, US

(73) Assignees: Johns Hopkins University, Baltimore, MD (US); OncoMethylome Sciences, S.A., Sart-Tilman (Liege) (BE)

(21) Appl. No.: 11/580,245

(22) Filed: Oct. 13, 2006

Related U.S. Application Data
(63) Continuation-in-part of application No. PCT/US06/14493, filed on Apr. 17, 2006.
(60) Provisional application No. 60/671,501, filed on Apr. 15, 2005.

(51) Int. Cl. A61K 31/704; C12Q 1/68; C12N 5/02; A61K 31/7088

(52) U.S. Cl. 514/34; 435/6; 435/375; 514/44

(57) ABSTRACT

Two hundred ten markers are provided which are epigenetically silenced in one or more cancer types. The markers can be used diagnostically, prognostically, therapeutically, and for selecting treatments that are well tailored for an individual patient. Restoration of expression of silenced genes can be useful therapeutically, for example, if the silenced gene is a tumor-suppressor gene. Restoration can be accomplished by supplying non-methylated copies of the silenced genes or polynucleotides encoding their encoded products. Alternatively, restoration can be accomplished using chemical demethylating agents or methylation inhibitors. Kits for testing for epigenetic silencing can be used in the context of diagnostics, prognostics, or for selecting “personalized medicine” treatments.

This application claims the benefit of U.S. provisional application Ser. No. 60/671,501, filed Apr. 15, 2005 and PCT application number PCT/US2006/01449, filed Apr. 17, 2006. The disclosures of these applications including the data submitted on CD-ROMs are incorporated herein by reference.

This application incorporates by reference the contents of each of two duplicate CD-ROMs. Each CD-ROM contains an identical 1,720 kB file labeled “882832_{—}1.txt” and containing the sequence listing for this application. Each CD-ROM also contains an identical 230 kB file labeled “882734_{—b 1.}txt” containing TABLE 9; an identical 33 kB file labeled “882733_{—}1.txt” containing TABLE 10; an identical 481 kB file labeled “882729_{—}1” containing TABLE 11; an identical 450 kB file labeled “882730_{—}1.txt” containing TABLE 12; an identical 2,458 kB file labeled “882732.txt” containing TABLE 13; and an identical 547 kB file labeled “882731_{—}1.txt” containing TABLE 14. The CD-ROMs were created on Apr. 11, 2006 and submitted with PCT/US2006/01449.

LENGTHY TABLES
The patent application contains a lengthy table section. A copy of the table is available in electronic form from the USPTO web site (http://seqdata.uspto.gov/?pageRequest=docDetail&DocID=US20090215709A1). An electronic copy of the table will also be available from the USPTO upon request and payment of the fee set forth in 37 CFR 1.19(b)(3).

### TECHNICAL FIELD OF THE INVENTION

This invention is related to the area of cancer diagnostics and therapeutics. In particular, it relates to aberrant methylation patterns of particular genes in cancers.

### BACKGROUND OF THE INVENTION

### DNA Methylation and its Role in Carcinogenesis

The information to make the cells of all living organisms is contained in their DNA. DNA is made up of a unique sequence of four bases: adenine (A), guanine (G), thymine (T) and cytosine (C). These bases are paired A to T and G to C on the two strands that form the DNA double helix. Strands of these pairs store information to make specific molecules grouped into regions called genes. Within each cell, there are processes that control what gene is turned on, or expressed, thus defining the unique function of the cell. One of these control mechanisms is provided by adding a methyl group onto cytosine (C). The methyl group tagged C can be written as mC.

DNA methylation plays an important role in determining whether some genes are expressed or not. By turning genes off that are not needed, DNA methylation is an essential control mechanism for the normal development and functioning of organisms. Alternatively, abnormal DNA methylation is one of the mechanisms underlying the changes observed with aging and development of many cancers.

Cancers have historically been linked to genetic changes caused by chromosomal mutations within the DNA. Mutations, hereditary or acquired, can lead to the loss of expression of genes critical for maintaining a healthy state. Evidence now supports that a relatively large number of cancers originate, not from mutations, but from inappropriate DNA methylation. In many cases, hyper-methylation of DNA incorrectly switches off critical genes, such as tumor suppressor genes or DNA repair genes, allowing cancers to develop and progress. This non-mutational process for controlling gene expression is described as epigenetics.

DNA methylation is a chemical modification of DNA performed by enzymes called methyltransferases, in which a methyl group (m) is added to certain cytosines (C) of DNA. This non-mutational (epigenetic) process (mC) is a critical factor in gene expression regulation. See, J. G. Herman, Seminars in Cancer Biology, 9: 359-67, 1999.

Although the phenomenon of gene methylation has attracted the attention of cancer researchers for some time, its true role in the progression of human cancers is just now being recognized. In normal cells, methylation occurs predominantly in regions of DNA that have few CG base repeats, while CpG islands, regions of DNA that have long repeats of CG bases, remain non-methylated. Gene promoter regions that control protein expression are often CpG island-rich. Aberrant methylation of these normally non-methylated CpG islands in the promoter region causes transcriptional inactivation or silencing of certain tumor suppressor expression in human cancers.

Genes that are hypermethylated in tumor cells are strongly specific to the tissue of origin of the tumor. Molecular signatures of cancers of all types can be used to improve cancer detection, the assessment of cancer risk and response to therapy. Promoter hypermethylation events provide some of the most promising markers for such purposes.

### Promoter Gene Hypermethylation: Promising Tumor Markers

Information regarding the hypermethylation of specific promoter genes can be beneficial to diagnosis, prognosis, and treatment of various cancers. Methylation of specific gene promoter regions can occur early and often in carcinogenesis making these markers ideal targets for cancer diagnostics.

Methylation patterns are tumor specific. Positive signals are always found in the same location of a gene. Real time PCR-based methods are highly sensitive, quantitative, and suitable for clinical use. DNA is stable and is found intact in readily available fluids (e.g., serum, sputum, stool and urine) and paraffin embedded tissues. Panels of pertinent gene markers may cover most human cancers.

### Diagnosis

Key to improving the clinical outcome in patients with cancer is diagnosis at its earliest stage, while it is still localized and readily treatable. The characteristics noted above provide the means for a more accurate screening and surveillance program by identifying higher-risk patients on a molecular basis. It could also provide justification-for more definitive follow up of patients who have molecular but not yet all the pathological or clinical features associated with malignancy.

### Predicting Treatment Response

Information about how a cancer develops through molecular events could allow a clinician to predict more accurately how such a cancer is likely to respond to specific chemotherapeutic agents. In this way, a regimen based on knowledge of the tumor's chemosensitivity could be rationally designed. Studies have shown that hypermethylation of the MGMT promoter in glioma patients is indicative of a good response to therapy, greater overall survival and a longer time to progression.

There is a continuing need in the art for new diagnostic markers and therapeutic targets for cancer to improve management of patient care.

### SUMMARY OF THE INVENTION

According to a first embodiment of the invention a method is provided for identifying a cell as neoplastic or predisposed to neoplasia. Epigenetic silencing of at least one gene listed in Table 5 is detected in a test cell. The test cell is identified as neoplastic or predisposed to neoplasia based on the detection of epigenetic silencing.

In another embodiment of the invention a method is provided for reducing or inhibiting neoplastic growth of a cell which exhibits epigenetic silenced transcription of at least one gene associated with a cancer. The cell may be a cervical, prostate, lung, breast, or colon cell. Expression of a polypeptide encoded by the epigenetic silenced gene is restored in the cell by contacting the cell with a CpG dinucleotide demethylating agent or with an agent that changes the histone acetylation status of cellular DNA or any other treatment affecting epigenetic mechanisms present in cells. The gene is selected from those listed in Table 5. Unregulated growth of the cell is thereby reduced or inhibited. If the cell is a breast or lung cell, the gene may or may not be APC. Expression of the gene is tested in the cell to monitor response to the demethylating or other epigenetic affecting agent.

Another aspect of the invention is a method of reducing or inhibiting neoplastic growth of a cell which exhibits epigenetic silenced transcription of at least one gene associated with a cancer. The cell may be a cervical prostate, lung, breast, or colon cell. A polynucleotide encoding a polypeptide is introduced into a cell which exhibits epigenetic silenced transcription of at least one gene listed in Table 5. The polypeptide is encoded by the epigenetic-silenced gene. The polypeptide is thereby expressed in the cell thereby restoring expression of the polypeptide in the cell. If the cell is a breast or lung cell, the gene may or may not be APC.

Still another aspect of the invention is a method of treating a cancer patient. The cancer may be a cervical prostate, lung, breast, or colon cell. A demethylating agent is administered to the patient in sufficient amounts to restore expression of a tumor-associated methylation-silenced gene selected from those listed in Table 5 in the patient's tumor. If the cancer is a breast or lung cancer, the gene may or may not be APC. Expression of the gene is tested in cancer cells of the patient to monitor response to the demethylating agent.

An additional embodiment of the invention provides a method of treating a cancer patient. The cancer may be a cervical, prostate, lung, breast, or colon cancer. A polynucleotide encoding a polypeptide is administered to the patient. The polypeptide is encoded by a gene listed in Table 5. The polypeptide is expressed in the patient's tumor thereby restoring expression of the polypeptide in the tumor. If the cancer is a breast or lung cancer, the gene may or may not be APC.

Yet another embodiment of the invention is a method for selecting a therapeutic strategy for treating a cancer patient. A gene selected from those listed in Table 5 whose expression in cancer cells of the patient is reactivated by a demethylating agent is identified. A therapeutic agent which reactivates expression of the gene is selected for treating the cancer patient. If the cancer cells are breast or lung cells, the gene may or may not be APC.

A further embodiment of the invention is a kit for assessing methylation in a cell sample. The kit comprises certain components in a package. One component is a reagent that (a) modifies methylated cytosine residues but not non-methylated cytosine residues, or that (b) modifies non-methylated cytosine residues but not methylated cytosine residues. A second component is a pair of oligonucleotide primers that specifically hybridizes under amplification conditions to a region of a gene selected from those listed in Table 5. The region is within about 1 kb of said gene's transcription start site.

These and other embodiments which will be apparent to those of skill in the art upon reading the specification provide the art with tools and methods for detection, diagnosis, therapy, and drug selection pertaining to neoplastic cells and cancers.

### BRIEF DESCRIPTION OF THE FIGURES

FIG. 1A-1B: Methylation specific PCR (MSP) for CCNA1 (FIG. 1A) and NPTX1 (FIG. 1B)

FIG. 2: Methylation specific PCR (MSP) for CEBPC and PODXL

FIG. 3A-FIG. 3F: Differential methylation for colorectal cells of markers OSMR (FIG. 3A), B4GALT1 (FIG. 3B), PAPSS2 (FIG. 3C), TUBG2 (FIG. 3D), SFRP4 (FIG. 3E), and NTRK2 (FIG. 3F)

FIG. 4. Table 1. Squamous lung cancer reactivated genes

FIG. 5. Table 2. Adenocarcinoma lung cancer reactivated genes

FIG. 6. Table 3. All lung cancer reactivated genes

FIG. 7. Table 4. Prostate cancer reactivated genes

FIG. 8A-8E. Table 5. All cancer reactivated genes

FIG. 9. Table 6. Breast cancer reactivated genes

FIG. 10A-10B. Table 7. Colorectal cancer reactivated genes

FIG. 11. Table 8. Cervical cancer reactivated genes

FIG. 12A-12D. Table 15. Correlation of transcript sequence to encoded protein sequence; also provides the order that the genes and proteins are listed in the sequence listing.

FIG. 13A-13B. Table 16. BSP results for cervical cancer tissues.

FIG. 14A-14R. Table 17. Correlation of transcript accession number to gene/protein name

FIG. 15. Table 18. Results for lung cancer tissues.

FIG. 16A-16B. Table 19. Results for breast cancer tissues

FIG. 17. Table 20. Results for colon cancer tissues.

FIG. 18. Table 21. Primers used for CMSP and OMSP for colon cells.

FIG. 19A-19B. Table 22. Primers used for various cancer types.

FIG. 20. Table 23. Methylation results in various cancer types

FIG. 21A-21C. FIG. 21A shows methylation data for OSMR in 100 pairs of colon tumor and normal cells. FIG. 21B shows B4GALT1 methylation in a variety of indicated tumor/normal pairs. FIG. 21C shows B4GALT1 methylation in additional tumor/normal pairs.

### BRIEF DESCRIPTION OF THE TABLES ON CD-R

Table 9. Combinations of two and three squamous lung cancer reactivated genes (on CD.)

Table 10. Combinations of two and three adenocarcinoma lung cancer reactivated genes (on CD)

Table 11. Combinations of two and three prostate cancer reactivated genes (on CD)

Table 12. Combinations of two and three breast cancer reactivated genes (on CD)

Table 13. Combinations of two and three colorectal cancer reactivated genes (on CD)

Table 14. Combinations of two and three cervical cancer reactivated genes (on CD)

### DETAILED DESCRIPTION OF THE INVENTION

The inventors have discovered a set of genes whose transcription is epigenetically silenced in cancers. All of the identified genes are shown in Table 5. Subsets which are associated with particular cancers are shown in Tables 1-4 and 6-8.

Epigenetic silencing of a gene can be determined by any method known in the art. One method is to determine that a gene which is expressed in normal cells is less expressed or not expressed in tumor cells. This method does not, on its own, however, indicate that the silencing is epigenetic, as the mechanism of the silencing could be genetic, for example, by somatic mutation. One method to determine that the silencing is epigenetic is to treat with a reagent, such as DAC (5′-deazacytidine), or with a reagent which changes the histone acetylation status of cellular DNA or any other treatment affecting epigenetic mechanisms present in cells, and observe that the silencing is reversed, i.e., that the expression of the gene is reactivated or restored. Another means to determine epigenetic silencing is to determine the presence of methylated CpG dinucleotide motifs in the silenced gene. Typically these reside near the transcription start site, for example, within about 1 kbp, within about 750 bp, or within about 500 bp.

Expression of a gene can be assessed using any means known in the art. Either mRNA or protein can be measured. Methods employing hybridization to nucleic acid probes can be employed for measuring specific mRNAs. Such methods include using nucleic acid probe arrays (microarray technology), in situ hybridization, and using Northern blots. Messenger RNA can also be assessed using amplification techniques, such as RT-PCR. Advances in genomic technologies now permit the simultaneous analysis of thousands of genes, although many are based on the same concept of specific probe-target hybridization. Sequencing-based methods are an alternative; these methods started with the use of expressed sequence tags (ESTs), and now include methods based on short tags, such as serial analysis of gene expression (SAGE) and massively parallel signature sequencing (MPSS). Differential display techniques provide yet another means of analyzing gene expression; this family of techniques is based on random amplification of cDNA fragments generated by restriction digestion, and bands that differ between two tissues identify cDNAs of interest. Specific proteins can be assessed using any convenient method including immunoassays and immuno-cytochemistry but are not limited to that. Most such methods will employ antibodies which are specific for the particular protein or protein fragments. The sequences of the mRNA (cDNA) and proteins of the markers of the present invention are provided in the sequence listing. The sequences are provided in the order of increasing accession numbers as shown in Table 15.

Methylation-sensitive restriction endonucleases can be used to detect methylated CpG dinucleotide motifs. Such endonucleases may either preferentially cleave methylated recognition sites relative to non-methylated recognition sites or preferentially cleave non-methylated relative to methylated recognition sites. Examples of the former are Acc III, Ban I, BstN I, Msp I, and Xma I. Examples of the latter are Acc II, Ava I, BssH II, BstU I, Hpa II, and Not I. Alternatively, chemical reagents can be used which selectively modify either the methylated or non-methylated form of CpG dinucleotide motifs.

Modified products can be detected directly, or after a further reaction which creates products which are easily distinguishable. Means which detect altered size and/or charge can be used to detect modified products, including but not limited to electrophoresis, chromatography, and mass spectrometry. Examples of such chemical reagents for selective modification include hydrazine and bisulfite ions. Hydrazine-modified DNA can be treated with piperidine to cleave it. Bisulfite ion-treated DNA can be treated with alkali.

A variety of amplification techniques may be used in a reaction for creating distinguishable products. Some of these techniques employ PCR. Other suitable amplification methods include the ligase chain reaction (LCR) (Barringer et al, 1990), transcription amplification (Kwoh et al. 1989; WO88/10315), selective amplification of target polynucleotide sequences (U.S. Pat. No. 6,410,276), consensus sequence primed polymerase chain reaction (U.S. Pat. No. 4,437,975), arbitrarily primed polymerase chain reaction (WO90/06995), nucleic acid based sequence amplification (NASBA) (U.S. Pat. Nos. 5,409,818; 5,554,517; 6,063,603), nick displacement amplification (WO2004/067726).

Sequence variation that reflects the methylation status at CpG dinucleotides in the original genomic DNA offers two approaches to PCR primer design. In the first approach, the primers do not themselves do not “cover” or hybridize to any potential sites of DNA methylation; sequence variation at sites of differential methylation are located between the two primers. Such primers are used in bisulphite genomic sequencing, COBRA, Ms-SNuPE. In the second approach, the primers are designed to anneal specifically with either the methylated or unmethylated version of the converted sequence. If there is a sufficient region of complementarity, e.g., 12, 15, 18, or 20 nucleotides, to the target, then the primer may also contain additional nucleotide residues that do not interfere with hybridization but may be useful for other manipulations. Exemplary of such other residues may be sites for restriction endonuclease cleavage, for ligand binding or for factor binding or linkers or repeats. The oligonucleotide primers may or may not be such that they are specific for modified methylated residues

One way to distinguish between modified and unmodified DNA is to hybridize oligonucleotide primers which specifically bind to one form or the other of the DNA. After hybridization, an amplification reaction can be performed and amplification products assayed. The presence of an amplification product indicates that a sample hybridized to the primer. The specificity of the primer indicates whether the DNA had been modified or not, which in turn indicates whether the DNA had been methylated or not. For example, bisulfite ions modify non-methylated cytosine bases, changing them to uracil bases. Uracil bases hybridize to adenine bases under hybridization conditions. Thus an oligonucleotide primer which comprises adenine bases in place of guanine bases would hybridize to the bisulfite-modified DNA, whereas an oligonucleotide primer containing the guanine bases would hybridize to the non-modified (methylated) cytosine residues in the DNA. Amplification using a DNA polymerase and a second primer yield amplification products which can be readily observed. Such a method is termed MSP (Methylation Specific PCR; U.S. Pat. Nos. 5,786,146; 6,017,704; 6,200,756). The amplification products can be optionally hybridized to specific oligonucleotide probes which may also be specific for certain products. Alternatively, oligonucleotide probes can be used which will hybridize to amplification products from both modified and nonmodified DNA.

Another way to distinguish between modified and nonmodified DNA is to use oligonucleotide probes which may also be specific for certain products. Such probes can be hybridized directly to modified DNA or to amplification products of modified DNA. Oligonucleotide probes can be labeled using any detection system known in the art. These include but are not limited to fluorescent moieties, radioisotope labeled moieties, bioluminescent moieties, luminescent moieties, chemiluminescent moieties, enzymes, substrates, receptors, or ligands.

Still another way for the identification of methylated CpG dinucleotides utilizes the ability of the MBD domain of the McCP2 protein to selectively bind to methylated DNA sequences (Cross et al, 1994; Shiraishi et al, 1999). Restriction enconuclease digested genomic DNA is loaded onto expressed His-tagged methyl-CpG binding domain that is immobilized to a solid matrix and used for preparative column chromatography to isolate highly methylated DNA sequences.

Real time chemistry allow for the detection of PCR amplification during the early phases of the reactions, and makes quantitation of DNA and RNA easier and more precise. A few variations of the real-time PCR are known. They include the TaqMan system and Molecular Beacon system which have separate probes labeled with a fluorophore and a fuorescence quencher. In the Scorpion system the labeled probe in the form of a hairpin structure is linked to the primer.

DNA methylation analysis has been performed successfully with a number of techniques which include the MALDI-TOFF, MassARRAY, MethyLight, Quantitative analysis of ethylated alleles (QAMA), enzymatic regional methylation assay (ERMA), HeavyMethyl, QBSUPT, MS-SNuPE, MethylQuant, Quantitative PCR sequencing, Oligonucleotide-based microarray, systems.

The number of genes whose silencing is tested and/or detected can vary: one, two, three, four, five, or more genes can be tested and/or detected. In some cases at least two genes are selected from one table selected from Tables 1-4 and 6-8. In other embodiments at least three genes are selected from one table selected from Tables 1-4 and 6-8.

If one or at least two genes are being tested and the cell is a prostate cell, at least one gene can be selected from the group consisting of CD3D, APOC1, NBL1, ING4, LEF1, CENTD3, MGC15396, FKBP4, PLTP, TFAP2A, ATXN1, BMP2, ENPEP, MCAM, SSBP2, PDLIM3, PAK3, B4GALT1, and NDP. More particularly, at least one gene can be selected from the group consisting of BMP2, ENPEP, MCAM, SSBP2, and NDP.

If one or at least two genes are being tested and the cell is a lung cell, at least one gene can be selected from the group consisting of PHKA2, CBR3, CAMK4, HOXB5, ZNF198, RGS4, RBM15B, PDLIM3, PAK3, PIGH, TUBB4, B4GALT1, and NISCH. More particularly, at least one gene can be selected from the group consisting of PAK3, PIGH, TUBB4, B4GALT1, and NISCH.

If one or at least two genes are being tested and the cell is a breast cell, at least one gene can be selected from the group consisting of BACH1, CKMT, GALE, HMG20B, KRT14, OGDHL, PON2, SESN1, KIF1A (kinesin family member 1A) PDLIM3 and MAL (T cell proliferation protein). More particularly, at least one gene can be elected from the group consisting of KIF1A (kinesin family member 1A) and MAL (T cell proliferation protein).

If one or at least two genes are being tested and the cell is a colon cell, at least one gene can be selected from the group consisting of B4GALT1, C10orf119, C10orf13, CBR1, COPS4, COVA1, CSRP1, DARS, DNAJC10, FKBP14, FN3KRP, GANAB, HUS1, KLF11, MRPL4, MYLK, NELF, NETO2, PAPSS2, RBMS2, RHOB, SECTM1, SIRT2, SIRT7, SLC35D1, SLC9A3R1, TTRAP, TUBG2, FLJ20277, MYBL2, GPR116, OSMR, PC4, SLC39A4, UBE3A, PDLIM3, NRTK2, OGDHL,

SFRP4, SSBP2, and UBE21. More particularly, at least one gene can be selected from the group consisting of GPR116, OSMR, PC4, SLC39A4, UBE3A, PDLIM3 and UBE21.

If one or at least two genes are being tested and the cell is a cervical cancer cell, at least one gene can be selected from the group consisting of PDCD4, TFPI2, ARMC7, TRM-HUMAN, OGDHL, PTGS2, CDK6, GPR39, HMGN2, C13ORF18, ASMTL, DLL4, NP-659450.1, NP-078820.1, CLU, HPCA, PLCG2, RALY, GNB4, CCNA1 NPTX1 and C90RF19. Particularly the at least one gene can be selected from the group consisting of TFPI2, ARMC7, TRM_HUMAN, OGDHL, PTGS2, GPR39, C13ORF18, ASMTL, CCNA1, NPTX1 and DLL4.

Testing can be performed diagnostically or in conjunction with a therapeutic regimen. Testing can be used to monitor efficacy of a therapeutic regimen, whether a chemotherapeutic agent or a biological agent, such as a polynucleotide.

Test cells for diagnostic, prognostic, or personalized medicine uses can be obtained from surgical samples, such as biopsies or fine needle aspirates, from paraffin embedded colon, rectum, breast, ovary, prostate, kidney, lung, brain on other organ tissues, from a body fluid such as bone marrow, blood, serum, lymph, cerebrospinal fluid, saliva, sputum, bronchial-lavage fluid, ductal fluids stool, urine, lymph nodes or semen. Such sources are not meant to be exhaustive, but rather exemplary. A test cell obtainable from such samples or fluids includes detached tumor cells or free nucleic acids that are released from dead tumor cells. Nucleic acids include RNA, genomic DNA, mitochondrial DNA, single or double stranded, and protein-associated nucleic acids. Any nucleic acid specimen in purified or non-purified form obtained from such test cell can be utilized as the starting nucleic acid or acids.

Demethylating agents can be contacted with cells in vitro or in vivo for the purpose of restoring normal gene expression to the cell. Suitable demethylating agents include, but are not limited to 5-aza-2′-deoxycytidine, 5-aza-cytidine, Zebularine, procaine, and L-ethionine. This reaction may be used for diagnosis, for determining predisposition, and for determining suitable therapeutic regimes. If the demethylating agent is used for treating colon, breast, lung, or prostate cancers, expression or methylation can be tested of a gene selected from the group consisting of CD3D, APOC1, NBL1,ING4, LEF1, CENTD3, MGC15396, FKBP4, PLTP, TFAP2A, ATXN1, BMP2, ENPEP, MCAM, SSBP2, PDLIM3, NDP, PHKA2, CBR3, CAMK4, HOXB5, ZNF198, RGS4, RBM15B, PDLIM3, PAK3, B4GALT1, PIGH, TUBB4, NISCH, BACH1, CKMT, GALE, HMG20B, KRT14, OGDHL, PON2, SESN1, KIF1A (kinesin family member 1A) PDLIM3, MAL (T cell proliferation protein) B4GALT1, C10orf119, C10orf13, CBR1, COPS4, COVA1, CSRP1, DARS, DNAJC10, FKBP14, FN3KRP, GANAB, HUS1, KLF11, MRPL4, MYLK, NELF, NETO2, PAPSS2, RBMS2, RHOB, SECTM1, SIRT2, SIRT7, SLC35D1, SLC9A3R1, TTRAP, TUBG2, FLJ20277, MYBL2, GPR116, OSMR, PC4, SLC39A4, UBE3A, PDLIM3 and UBE21. If the cell is a prostate cell, the gene can be selected from the group consisting of BMP2, ENPEP, MCAM, SSBP2, PAK3, B4GALT1, and NDP. If the cell is a lung cell, the gene can be selected from the group consisting of PAK3, PIGH, TUBB4, B4GALT1, and NISCH. If the cell is a breast cell, the gene can be selected from the group consisting of KIF1A (kinesin family member 1A) and MAL (T cell proliferation protein). If the cell is a colon cell, the gene can be selected from the group consisting of GPR116, OSMR, PC4, SLC39A4, UBE3A, PDLIM3, NRTK2, OGDHL, SFRP4, SSBP2, and UBE21. If the cell is a cervical cancer cell, at least one gene can be selected from the group consisting of PDCD4, TFPI2, ARMC7, TRM-HUMAN, OGDHL, PTGS2, CDK6, GPR39, HMGN2, C13ORF18, ASMTL, DLL4, NP-659450.1, NP-078820.1, CLU, HPCA, PLCG2, RALY, GNB4, CCNA1 NPTX1 and C90RF19. Particularly the at least one gene can be selected from the group consisting of TFPI2, ARMC7, TRM_HUMAN, OGDHL, PTGS2, GPR39, C13ORF18, ASMTL, CCNA1, NPTX1 and DLL4.

An alternative way to restore epigenetically silenced gene expression is to introduce a non-methylated polynucleotide into a cell, so that it will be expressed in the cell. Various gene therapy vectors and vehicles are known in the art and any can be used as is suitable for a particular situation. Certain vectors are suitable for short term expression and certain vectors are suitable for prolonged expression. Certain vectors are trophic for certain organs and these can be used as is appropriate in the particular situation. Vectors may be viral or non-viral. The polynucleotide can, but need not, be contained in a vector, for example, a viral vector, and can be formulated, for example, in a matrix such as a liposome, microbubbles. The polynucleotide can be introduced into a cell by administering the polynucleotide to the subject such that it contacts the cell and is taken up by the cell and the encoded polypeptide expressed. Suitable polynucleotides are provided in the sequence listing SEQ ID NO: 1-210. Polynucleotides encoding the polypeptides shown in SEQ ID NO: 211-420 can also be used. Preferably the specific polynucleotide will be one which the patient has been tested for and been found to carry a silenced version. The polynucleotides for treating colon, breast, lung, or prostate cancers will typically encode a gene selected from the group consisting of CD3D, APOC1, NBL1, ING4, LEF1, CENTD3, MGC15396, FKBP4, PLTP, TFAP2A, ATXN1, BMP2, ENPEP, MCAM, SSBP2, PDLIM3, NDP, PHKA2, CBR3, CAMK4, HOXB5, ZNF198, RGS4, RBM15B, PDLIM3, PAK3, PIGH, TUBB4, NISCH, BACH1, CKMT, GALE, HMG20B, KRT14, OGDHL, PON2, SESN1, KIF1A (kinesin family member 1A) PDLIM3, MAL (T cell proliferation protein) B4GALT1, C10orf119, C10orf13, CBR1, COPS4, COVA1, CSRP1, DARS, DNAJC10, FKBP14, FN3KRP, GANAB, HUS1, KLF11, MRPL4, MYLK, NELF, NETO2, PAPSS2, RBMS2, RHOB, SECTM1, SIRT2, SIRT7, SLC35D1, SLC9A3R1, TTRAP, TUBG2, FLJ20277, MYBL2, GPR116, OSMR, PC4, SLC39A4, UBE3A, PDLIM3 and UBE21. If the cell is a prostate cell, the gene can be selected from the group consisting of BMP2, ENPEP, MCAM, SSBP2, PAK3, B4GALT1, and NDP. If the cell is a lung cell, the gene can be selected from the group consisting of PAK3, PIGH, TUBB4, B4GALT1, and NISCH. If the cell is a breast cell, the gene can be selected from the group consisting of KIF1A (kinesin family member 1A) and MAL (T cell proliferation protein). If the cell is a colon cell, the gene can be selected from the group consisting of GPR116, OSMR, PC4, SLC39A4, UBE3A, PDLIM3, NRTK2, OGDHL, SFRP4, SSBP2, and UBE21. If the cell is a cervical cancer cell, at least one gene can be selected from the group consisting of PDCD4, TFPI2, ARMC7, TRM-HUMAN, OGDHL, PTGS2, CDK6, GPR39, HMGN2, C130RF18, ASMTL, DLL4, NP-659450.1, NP-078820.1, CLU, HPCA, PLCG2, RALY, GNB4, CCNA1 NPTX1 and C9ORF19. Particularly the at least one gene can be selected from the group consisting of TFPI2, ARMC7, TRM_HUMAN, OGDHL, PTGS2, GPR39, C13ORF18, ASMTL, CCNA1, NPTX1 and DLL4.

Cells exhibiting methylation silenced gene expression generally are contacted with the demethylating agent in vivo by administering the agent to a subject. Where convenient, the demethylating agent can be administered using, for example, a catheterization procedure, at or near the site of the cells exhibiting unregulated growth in the subject, or into a blood vessel in which the blood is flowing to the site of the cells. Similarly, where an organ, or portion thereof, to be treated can be isolated by a shunt procedure, the agent can be administered via the shunt, thus substantially providing the agent to the site containing the cells. The agent also can be administered systemically or via other routes known in the art.

The polynucleotide can include, in addition to polypeptide coding sequence, operatively linked transcriptional regulatory elements, translational regulatory elements, and the like, and can be in the form of a naked DNA molecule, which can be contained in a vector, or can be formulated in a matrix such as a liposome or microbubbles that facilitates entry of the polynucleotide into the particular cell. The term “operatively linked” refers to two or more molecules that are positioned with respect to each other such that they act as a single unit and effect a function attributable to one or both molecules or a combination thereof. A polynucleotide sequence encoding a desired polypeptide can be operatively linked to a regulatory element, in which case the regulatory element confers its regulatory effect on the polynucleotide similar to the way in which the regulatory element would affect a polynucleotide sequence with which it normally is associated with in a cell.

The polynucleotide encoding the desired polypeptide to be administered to a mammal or a human or to be contacted with a cell may contain a promoter sequence, which can provide constitutive or, if desired, inducible or tissue specific or developmental stage specific expression of the polynucleotide, a poly-A recognition sequence, and a ribosome recognition site or internal ribosome entry site, or other regulatory elements such as an enhancer, which can be tissue specific. The vector also may contain elements required for replication in a prokaryotic or eukaryotic host system or both, as desired. Such vectors, which include plasmid vectors and viral vectors such as bacteriophage, baculovirus, retrovirus, lentivirus, adenovirus, vaccinia virus, semliki forest virus and adeno-associated virus vectors, are well known and can be purchased from a commercial source (Promega, Madison Wis.; Stratagene, La Jolla Calif.; GIBCO/BRL, Gaithersburg Md.) or can be constructed by one skilled in the art (see, for example, Meth. Enzymol., Vol. 185, Goeddel, ed. (Academic Press, Inc., 1990); Jolly, Canc. Gene Ther. 1:51-64, 1994; Flotte, J. Bioenerg. Biomemb. 25:37-42, 1993; Kirshenbaum et al., J. Clin. Invest. 92:381-387, 1993; each of which is incorporated herein by reference).

A tetracycline (tet) inducible promoter can be used for driving expression of a polynucleotide encoding a desired polypeptide. Upon administration of tetracycline, or a tetracycline analog, to a subject containing a polynucleotide operatively linked to a tet inducible promoter, expression of the encoded polypeptide is induced. The polynucleotide alternatively can be operatively linked to tissue specific regulatory element, for example, a liver cell specific regulatory element such as an α-fetoprotein promoter (Kanai et al., Cancer Res. 57:461-465, 1997; He et al., J. Exp. Clin. Cancer Res. 19:183-187, 2000) or an albumin promoter (Power et al., Biochem. Biophys. Res. Comm. 203:1447-1456, 1994; Kuriyama et al., Int. J. Cancer 71:470-475, 1997); a muscle cell specific regulatory element such as a myoglobin promoter (Devlin et al., J. Biol. Chem. 264:13896-13901, 1989; Yan et al., J. Biol. Chem. 276:17361-17366, 2001); a prostate cell specific regulatory element such as the PSA promoter (Schuur et al., J. Biol. Chem. 271:7043-7051, 1996; Latham et al., Cancer Res. 60:334-341, 2000); a pancreatic cell specific regulatory element such as the elastase promoter (Ornitz et al., Nature 313:600-602, 1985; Swift et al., Genes Devel. 3:687-696, 1989); a leukocyte specific regulatory element such as the leukosialin (CD43) promoter (Shelley et al., Biochem. J. 270:569-576, 1990; Kudo and Fukuda, J. Biol. Chem. 270:13298-13302, 1995); or the like, such that expression of the polypeptide is restricted to particular cell in an individual, or to particular cells in a mixed population of cells in culture, for example, an organ culture. Regulatory elements, including tissue specific regulatory elements, many of which are commercially available, are well known in the art (see, for example, InvivoGen; San Diego Calif.).

Viral expression vectors can be used for introducing a polynucleotide into a cell, particularly a cell in a subject. Viral vectors provide the advantage that they can infect host cells with relatively high efficiency and can infect specific cell types. For example, a polynucleotide encoding a desired polypeptide can be cloned into a baculovirus vector, which then can be used to infect an insect host cell, thereby providing a means to produce large amounts of the encoded polypeptide. Viral vectors have been developed for use in particular host systems, particularly mammalian systems and include, for example, retroviral vectors, other lentivirus vectors such as those based on the human immunodeficiency virus (HIV), adenovirus vectors, adeno-associated virus vectors, herpesvirus vectors, hepatitis virus vectors, vaccinia virus vectors, and the like (see Miller and Rosman, BioTechniques 7:980-990, 1992; Anderson et al., Nature 392:25-30 Suppl., 1998; Verma and Somia, Nature 389:239-242, 1997; Wilson, New Engl. J. Med. 334:1185-1187 (1996), each of which is incorporated herein by reference).

A polynucleotide, which can optionally be contained in a vector, can be introduced into a cell by any of a variety of methods known in the art (Sambrook et al., supra, 1989; Ausubel et al., Current Protocols in Molecular Biology, John Wiley and Sons, Baltimore, Md. (1987, and supplements through 1995), each of which is incorporated herein by reference). Such methods include, for example, transfection, lipofection, microinjection, electroporation and, with viral vectors, infection; and can include the use of liposomes, microemulsions or the like, which can facilitate introduction of the polynucleotide into the cell and can protect the polynucleotide from degradation prior to its introduction into the cell. A particularly useful method comprises incorporating the polynucleotide into microbubbles, which can be injected into the circulation. An ultrasound source can be positioned such that ultrasound is transmitted to the tumor, wherein circulating microbubbles containing the polynucleotide are disrupted at the site of the tumor due to the ultrasound, thus providing the polynucleotide at the site of the cancer. The selection of a particular method will depend, for example, on the cell into which the polynucleotide is to be introduced, as well as whether the cell is in culture or in situ in a body.

Introduction of a polynucleotide into a cell by infection with a viral vector can efficiently introduce the nucleic acid molecule into a cell. Moreover, viruses are very specialized and can be selected as vectors based on an ability to infect and propagate in one or a few specific cell types. Thus, their natural specificity can be used to target the nucleic acid molecule contained in the vector to specific cell types. A vector based on an HIV can be used to infect T cells, a vector based on an adenovirus can be used, for example, to infect respiratory epithelial cells, a vector based on a herpesvirus can be used to infect neuronal cells, and the like. Other vectors, such as adeno-associated viruses can have greater host cell range and, therefore, can be used to infect various cell types, although viral or non-viral vectors also can be modified with specific receptors or ligands to alter target specificity through receptor mediated events. A polynucleotide of the invention, or a vector containing the polynucleotide can be contained in a cell, for example, a host cell, which allows propagation of a vector containing the polynucleotide, or a helper cell, which allows packaging of a viral vector containing the polynucleotide. The polynucleotide can be transiently contained in the cell, or can be stably maintained due, for example, to integration into the cell genome.

A polypeptide according to any of SEQ ID NO: 211-420 can be administered directly to the site of a cell exhibiting unregulated growth in the subject. The polypeptide can be produced and isolated, and formulated as desired, using methods as disclosed herein, and can be contacted with the cell such that the polypeptide can cross the cell membrane of the target cells. The polypeptide may be provided as part of a fusion protein, which includes a peptide or polypeptide component that facilitates transport across cell membranes. For example, a human immunodeficiency virus (HIV) TAT protein transduction domain or a nuclear localization domain may be fused to the marker of interest. The administered polypeptide can be formulated in a matrix that facilitates entry of the polypeptide into a cell.

An agent such as a demethylating agent, a polynucleotide, or a polypeptide is typically formulated in a composition suitable for administration to the subject. Thus, the invention provides compositions containing an agent that is useful for restoring regulated growth to a cell exhibiting unregulated growth due to methylation silenced transcription of one or more genes. The agents are useful as medicaments for treating a subject suffering from a pathological condition associated with such unregulated growth. Such medicaments generally include a carrier. Acceptable carriers are well known in the art and include, for example, aqueous solutions such as water or physiologically buffered saline or other solvents or vehicles such as glycols, glycerol, oils such as olive oil or injectable organic esters. An acceptable carrier can contain physiologically acceptable compounds that act, for example, to stabilize or to increase the absorption of the conjugate. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know or readily be able to determine an acceptable carrier, including a physiologically acceptable compound. The nature of the carrier depends on the physico-chemical characteristics of the therapeutic agent and on the route of administration of the composition. Administration of therapeutic agents or medicaments can be by the oral route or parenterally such as intravenously, intramuscularly, subcutaneously, transdermally, intranasally, intrabronchially, vaginally, rectally, intratumorally, or other such method known in the art. The pharmaceutical composition also can contain one more additional therapeutic agents.

The therapeutic agents can be incorporated within an encapsulating material such as into an oil-in-water emulsion, a microemulsion, micelle, mixed micelle, liposome, microsphere, microbubbles or other polymer matrix (see, for example, Gregoriadis, Liposome Technology, Vol. 1 (CRC Press, Boca Raton, Fla. 1984); Fraley, et al., Trends Biochem. Sci., 6:77 (1981), each of which is incorporated herein by reference). Liposomes, for example, which consist of phospholipids or other lipids, are nontoxic, physiologically acceptable and metabolizable carriers that are relatively simple to make and administer. “Stealth” liposomes (see, for example, U.S. Pat. Nos. 5,882,679; 5,395,619; and 5,225,212, each of which is incorporated herein by reference) are an example of such encapsulating materials particularly useful for preparing a composition useful in a method of the invention, and other “masked” liposomes similarly can be used, such liposomes extending the time that the therapeutic agent remain in the circulation. Cationic liposomes, for example, also can be modified with specific receptors or ligands (Morishita et al., J. Clin. Invest., 91:2580-2585 (1993), which is incorporated herein by reference). In addition, a polynucleotide agent can be introduced into a cell using, for example, adenovirus-polylysine DNA complexes (see, for example, Michael et al., J. Biol. Chem. 268:6866-6869 (1993), which is incorporated herein by reference).

The route of administration of the composition containing the therapeutic agent will depend, in part, on the chemical structure of the molecule. Polypeptides and polynucleotides, for example, are not efficiently delivered orally because they can be degraded in the digestive tract. However, methods for chemically modifying polypeptides, for example, to render them less susceptible to degradation by endogenous proteases or more absorbable through the alimentary tract may be used (see, for example, Blondelle et al., supra, 1995; Ecker and Crook, supra, 1995).

The total amount of an agent to be administered in practicing a method of the invention can be administered to a subject as a single dose, either as a bolus or by infusion over a relatively short period of time, or can be administered using a fractionated treatment protocol, in which multiple doses are administered over a prolonged period of time. One skilled in the art would know that the amount of the composition to treat a pathologic condition in a subject depends on many factors including the age and general health of the subject as well as the route of administration and the number of treatments to be administered. In view of these factors, the skilled artisan would adjust the particular dose as necessary. In general, the formulation of the composition and the routes and frequency of administration are determined, initially, using Phase I and Phase II clinical trials.

The composition can be formulated for oral formulation, such as a tablet, or a solution or suspension form; or can comprise an admixture with an organic or inorganic carrier or excipient suitable for enteral or parenteral applications, and can be compounded, for example, with the usual non-toxic, pharmaceutically acceptable carriers for tablets, pellets, capsules, suppositories, solutions, emulsions, suspensions, or other form suitable for use. The carriers, in addition to those disclosed above, can include glucose, lactose, mannose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening or coloring agents and perfumes can be used, for example a stabilizing dry agent such as triulose (see, for example, U.S. Pat. No. 5,314,695).

Although accuracy and sensitivity may be achieved by using a combination of markers, such as 5 or 6 markers, practical considerations may dictate use of smaller combinations. Any combination of markers for a specific cancer may be used which comprises 2, 3, 4, or 5 markers. Each of the combinations for two and three markers are listed in attached Tables found on CD-ROM. Other combinations of four or five markers can be readily envisioned given the specific disclosures of individual markers provided herein.

The level of methylation of the differentially methylated GpG islands can provide a variety of information a about the disease or cancer. It can be used to diagnose a disease or cancer in the individual. Alternatively, it can be used to predict the course of the disease or cancer in the individual or to predict the suspectibility to disease or cancer or to stage the progression of the disease or cancer in the individual. Otherwise, it can help to predict the likelihood of overall survival or predict the likelihood of reoccurrence of disease or cancer and to determine the effectiveness of a treatment course undergone by the individual. Increase or decrease of methylation levels in comparison with reference level and alterations in the increase/decrease when detected provide useful prognostic and diagnostic value.

The prognostic methods can be used to identify surgically treated patients likely to experience cancer reoccurrence. Such patients can be offered additional therapeutic options, including pre-operative or post-operative options such as chemotherapy, radiation, biological modifiers, or other therapies.

A therapeutic strategy for treating a prostate, lung, breast, or colon cancer patient can be selected based on reactivation of epigenetically silenced genes. First a gene selected from those listed in Table 5 is identified whose expression in cancer cells of the patient is reactivated by a demethylating agent. Then a therapeutic agent is selected which reactivates expression of the gene. If the cancer cells are breast or lung cells, the gene is not APC. If the cell is a prostate cell, a lung cell, a breast cell or a colon cell, the gene can be selected from the group consisting of CD3D, APOC1, NBL1, ING4, LEF1, CENTD3, MGC15396, FKBP4, PLTP, TFAP2A, ATXN1, BMP2, ENPEP, MCAM, SSBP2, PDLIM3, NDP, PHKA2, CBR3, CAMK4, HOXB5, ZNF198, RGS4, RBM15B, PDLIM3, PAK3, PIGH, TUBB4, NISCH, BACH1, CKMT, GALE, HMG20B, KRT14, OGDHL, PON2, SESN1, KIF1A (kinesin family member 1A) PDLIM3, MAL (T cell proliferation protein) B4GALT1, C10orf119, C10orf13, CBR1, COPS4, COVA1, CSRP1, DARS, DNAJC10, FKBP14, FN3KRP, GANAB, HUS1, KLF11, MRPL4, MYLK, NELF, NETO2, PAPSS2, RBMS2, RHOB, SECTM1, SIRT2, SIRT7, SLC35D1, SLC9A3R1, TTRAP, TUBG2, FLJ20277, MYBL2, GPR116, OSMR, PC4, SLC39A4, UBE3A, PDLIM3, NRTK2, SFRP4, SSBP2, and UBE21. More particularly, the gene can be selected from the group consisting of BMP2, ENPEP, MCAM, SSBP2, NDP, PAK3, PIGH, TUBB4, and NISCH. KIF1A (kinesin family member 1A), MAL (T cell proliferation protein), GPR116, OSMR, PC4, SLC39A4, UBE3A, PDLIM3, NRTK2, OGDHL, SFRP4, SSBP2, and UBE21. If the cancer is prostate cancer, the gene can be selected from the group consisting of BMP2, ENPEP, MCAM, SSBP2, PAK3, B4GALT1, and NDP. If the cancer is lung cancer, the gene can be selected from the group consisting of PAK3, PIGH, TUBB4, B4GALT1, and NISCH. If the cancer is breast cancer, the gene can be selected from the group consisting of KIF1A (kinesin family member 1A) and MAL (T cell proliferation protein). If the cancer is colon cancer, the gene can be selected from the group consisting of GPR116, OSMR, PC4, SLC39A4, UBE3A, PDLIM3, and UBE21. If the cell is a cervical cancer cell, at least one gene can be selected from the group consisting of PDCD4, TFPI2, ARMC7, TRM-HUMAN, OGDHL, PTGS2, CDK6, GPR39, HMGN2, C130RF18, ASMTL, DLL4, NP-659450.1, NP-078820.1, CLU, HPCA, PLCG2, RALY, GNB4, CCNA1 NPTX1 and C90RF19. Particularly the at least one gene can be selected from the group consisting of TFPI2, ARMC7, TRM_HUMAN, OGDHL, PTGS2, GPR39, C13ORF18, ASMTL, CCNA1, NPTX1 and DLL4.

Kits according to the present invention are assemblages of reagents for testing methylation. They are typically in a package which contains all elements, optionally including instructions. The package may be divided so that components are not mixed until desired. Components may be in different physical states. For example, some components may be lyophilized and some in aqueous solution. Some may be frozen. Individual components may be separately packaged within the kit. The kit may contain reagents, as described above for differentially modifying methylated and non-methylated cytosine residues. Desirably the kit will contain oligonucleotide primers which specifically hybridize to regions within 1 kb of the transcription start sites of the genes/markers identified in the attached Table 5. Typically the kit will contain both a forward and a reverse primer for a single gene or marker. If there is a sufficient region of complementarity, e.g., 12, 15, 18, or 20 nucleotides, then the primer may also contain additional nucleotide residues that do not interfere with hybridization but may be useful for other manipulations. Exemplary of such other residues may be sites for restriction endonuclease cleavage, for ligand binding or for factor binding or linkers or repeats. The oligonucleotide primers may or may not be such that they are specific for modified methylated residues. The kit may optionally contain oligonucleotide probes. The probes may be specific for sequences containing modified methylated residues or for sequences containing non-methylated residues. The kit may optionally contain reagents for modifying methylated cytosine residues. The kit may also contain components for performing amplification, such as a DNA polymerase and deoxyribonucleotides. Means of detection may also be provided in the kit, including detectable labels on primers or probes. Kits may also contain reagents for detecting gene expression for one of the markers of the present invention (Table 5). Such reagents may include probes, primers, or antibodies, for example. In the case of enzymes or ligands, substrates or binding partners may be sued to assess the presence of the marker.

In one aspect of this embodiment, the gene is contacted with hydrazine, which modifies cytosine residues, but not methylated cytosine residues, then the hydrazine treated gene sequence is contacted with a reagent such as piperidine, which cleaves the nucleic acid molecule at hydrazine modified cytosine residues, thereby generating a product comprising fragments. By separating the fragments according to molecular weight, using, for example, an electrophoretic, chromatographic, or mass spectrographic method, and comparing the separation pattern with that of a similarly treated corresponding non-methylated gene sequence, gaps are apparent at positions in the test gene contained methylated cytosine residues. As such, the presence of gaps is indicative of methylation of a cytosine residue in the CpG dinucleotide in the target gene of the test cell.

Bisulfite ions, for example, sodium bisulfite, convert non-methylated cytosine residues to bisulfite modified cytosine residues. The bisulfite ion treated gene sequence can be exposed to alkaline conditions, which convert bisulfite modified cytosine residues to uracil residues. Sodium bisulfite reacts readily with the 5,6-double bond of cytosine (but poorly with methylated cytosine) to form a sulfonated cytosine reaction intermediate that is susceptible to deamination, giving rise to a sulfonated uracil. The sulfonate group can be removed by exposure to alkaline conditions, resulting in the formation of uracil. The DNA can be amplified, for example, by PCR, and sequenced to determine whether CpG sites are methylated in the DNA of the sample. Uracil is recognized as a thymine by Taq polymerase and, upon PCR, the resultant product contains cytosine only at the position where 5-methylcytosine was present in the starting template DNA. One can compare the amount or distribution of uracil residues in the bisulfite ion treated gene sequence of the test cell with a similarly treated corresponding non-methylated gene sequence. A decrease in the amount or distribution of uracil residues in the gene from the test cell indicates methylation of cytosine residues in CpG dinucleotides in the gene of the test cell. The amount or distribution of uracil residues also can be detected by contacting the bisulfite ion treated target gene sequence, following exposure to alkaline conditions, with an oligonucleotide that selectively hybridizes to a nucleotide sequence of the target gene that either contains uracil residues or that lacks uracil residues, but not both, and detecting selective hybridization (or the absence thereof) of the oligonucleotide.

Any marker can be used for testing lung, prostate, breast or colon cells selected from the group consisting of CD3D, APOC1, NBL1, ING4, LEF1, CENTD3, MGC15396, FKBP4, PLTP, TFAP2A, ATXN1, BMP2, ENPEP, MCAM, SSBP2, PDLIM3, NDP, PHKA2, CBR3, CAMK4, HOXB5, ZNF198, RGS4, RBM15B, PDLIM3, PAK3, PIGH, TUBB4, NISCH, BACH1, CKMT, GALE, HMG20B, KRT14, OGDHL, PON2, SESN1, KIF1A (kinesin family member 1A) PDLIM3, MAL (T cell proliferation protein) B4GALT1, C10orf119, C10orf13, CBR1, COPS4, COVA1, CSRP1, DARS, DNAJC10, FKBP14, FN3KRP, GANAB, HUS1, KLF11, MRPL4, MYLK, NELF, NETO2, PAPSS2, RBMS2, RHOB, SECTM1, SIRT2, SIRT7, SLC35D1, SLC9A3R1, TTRAP, TUBG2, FLJ20277, MYBL2, GPR116, OSMR, PC4, SLC39A4, UBE3A, PDLIM3, NRTK2, SFRP4, SSBP2, and UBE21. Markers which are useful for prostate cancer are CD3D, APOC1, NBL1, ING4, LEF1, CENTD3, MGC15396, FKBP4, PLTP, TFAP2A, ATXN1, BMP2, ENPEP, MCAM, SSBP2, PDLIM3, PAK3, B4GALT1, and NDP. Particularly useful among these are BMP2, ENPEP, MCAM, SSBP2, and NDP. Markers which are useful for lung cancer are PHKA2, CBR3, CAMK4, HOXB5, ZNF198, RGS4, RBM15B, PDLIM3, PAK3, PIGH, TUBB4, B4GALT1, and NISCH. Particularly useful among these are PAK3, PIGH, TUBB4, B4GALT1, and NISCH. Markers which are useful for breast cancer are BACH1, CKMT, GALE, HMG20B, KRT14, OGDHL, PON2, SESN1, KIF1A (kinesin family member 1A) PDLIM3 and MAL (T cell proliferation protein). Particularly useful among these are KIF1A (kinesin family member 1A) and MAL (T cell proliferation protein). Markers which are useful for colon cancer are B4GALT1, C10orf119, C10orf13, CBR1, COPS4, COVA1, CSRP1, DARS, DNAJC10, FKBP14, FN3KRP, GANAB, HUS1, KLF11, MRPL4, MYLK, NELF, NETO2, PAPSS2, RBMS2, RHOB, SECTM1, SIRT2, SIRT7, SLC35D1, SLC9A3R1, TTRAP, TUBG2, FLJ20277, MYBL2, GPR116, OSMR, PC4, SLC39A4, UBE3A, PDLIM3, NRTK2, OGDHL, SFRP4, SSBP2, and UBE21. Particularly useful among these are GPR116, OSMR, PC4, SLC39A4, UBE3A, PDLIM3 and UBE21. If the cell is a cervical cancer cell, at least one gene can be selected from the group consisting of PDCD4, TFPI2, ARMC7, TRM-HUMAN, OGDHL, PTGS2, CDK6, GPR39, HMGN2, C13ORF18, ASMTL, DLL4, NP-659450.1, NP-078820.1, CLU, HPCA, PLCG2, RALY, GNB4, CCNA1 NPTX1 and C90RF19. Particularly the at least one gene can be selected from the group consisting of TFPI2, ARMC7, TRM_HUMAN, OGDHL, PTGS2, GPR39, C13ORF18, ASMTL, CCNA1, NPTX1 and DLL4.

Test compounds can be tested for their potential to treat cancer. Cancer cells for testing can be selected from the group consisting of prostate, lung, breast, and colon cancer. Expression of a gene selected from those listed in Table 5 is determined and if it is increased by the compound in the cell or if methylation of the gene is decreased by the compound in the cell, one can identify it as having potential as a treatment for cancer. For this purpose, the gene can be selected from the group consisting of CD3D, APOC1, NBL1, ING4, LEF1, CENTD3, MGC15396, FKBP4, PLTP, TFAP2A, ATXN1, BMP2, ENPEP, MCAM, SSBP2, PDLIM3, NDP, PHKA2, CBR3, CAMK4, HOXB5, ZNF198, RGS4, RBM15B, PDLIM3, PAK3, PIGH, TUBB4, NISCH, BACH1, CKMT, GALE, HMG20B, KRT14, OGDHL, PON2, SESN1, KIF1A (kinesin family member 1A) PDLIM3, MAL (T cell proliferation protein) B4GALT1, C10orf119, C10orf13, CBR1, COPS4, COVA1, CSRP1, DARS, DNAJC10, FKBP14, FN3KRP, GANAB, HUS1, KLF11, MRPL4, MYLK, NELF, NETO2, PAPSS2, RBMS2, RHOB, SECTM1, SIRT2, SIRT7, SLC35D1, SLC9A3R1, TTRAP, TUBG2, FLJ20277, MYBL2, GPR116, OSMR, PC4, SLC39A4, UBE3A, PDLIM3, NRTK2, SFRP4, SSBP2, and UBE21. More particularly, the gene can be selected from the group consisting of BMP2, ENPEP, MCAM, SSBP2, NDP, PAK3, PIGH, TUBB4, and NISCH. KIF1A (kinesin family member 1A), MAL (T cell proliferation protein), GPR116, OSMR, PC4, SLC39A4, UBE3A, PDLIM3 and UBE21. If the cell is a prostate cell, the gene can be selected from the group consisting of BMP2, ENPEP, MCAM, SSBP2, PAK3, B4GALT1, and NDP. If the cell is a lung cell, the gene can be selected from the group consisting of PAK3, PIGH, TUBB4, B4GALT1, and NISCH. If the cell is a breast cell, the gene can be selected from the group consisting of KIF1A (kinesin family member 1A) and MAL (T cell proliferation protein). If the cell is a colon cell, the gene can be selected from the group consisting of GPR116, OSMR, PC4, SLC39A4, UBE3A, PDLIM3, NRTK2, OGDHL, SFRP4, SSBP2, and UBE21. If the cell is a cervical cancer cell, at least one gene can be selected from the group consisting of PDCD4, TFPI2, ARMC7, TRM-HUMAN, OGDHL, PTGS2, CDK6, GPR39, HMGN2, C13ORF18, ASMTL, DLL4, NP-659450.1, NP-078820.1, CLU, HPCA, PLCG2, RALY, GNB4, CCNA1 NPTX1 and C90RF19. Particularly the at least one gene can be selected from the group consisting of TFPI2, ARMC7, TRM_HUMAN, OGDHL, PTGS2, GPR39, C130RF18, ASMTL, CCNA1, NPTX1 and DLL4.

Alternatively such tests can be used to determine a prostate, lung, breast, or colon cancer patient's response to a chemotherapeutic agent. The patient can be treated with a chemotherapeutic agent. If expression of a gene selected from those listed in Table 5 is increased by the compound in cancer cells or if methylation of the gene is decreased by the compound in cancer cells it can be selected as useful for treatment of the patient. If the patient has cancer cells which are prostate, a lung, a breast, or colon, the gene can be selected from the group consisting of CD3D, APOC1, NBL1, ING4, LEF1, CENTD3, MGC15396, FKBP4, PLTP, TFAP2A, ATXN1, BMP2, ENPEP, MCAM, SSBP2, PDLIM3, NDP, PHKA2, CBR3, CAMK4, HOXB5, ZNF198, RGS4, RBM15B, PDLIM3, PAK3, PIGH, TUBB4, NISCH, BACH1, CKMT, GALE, HMG20B, KRT14, OGDHL, PON2, SESN1, KIF1A (kinesin family member 1A) PDLIM3, MAL (T cell proliferation protein) B4GALT1, C10orf119, C10orf13, CBR1, COPS4, COVA1, CSRP1, DARS, DNAJC10, FKBP14, FN3KRP, GANAB, HUS1, KLF11, MRPL4, MYLK, NELF, NETO2, PAPSS2, RBMS2, RHOB, SECTM1, SIRT2, SIRT7, SLC35D1, SLC9A3R1, TTRAP, TUBG2, FLJ20277, MYBL2, GPR116, OSMR, PC4, SLC39A4, UBE3A, PDLIM3, NRTK2, SFRP4, SSBP2, and UBE21. More particularly, the marker or gene can be selected from the group consisting of BMP2, ENPEP, MCAM, SSBP2, NDP, PAK3, PIGH, TUBB4, and NISCH. KIF1A (kinesin family member 1A), MAL (T cell proliferation protein), GPR116, OSMR, PC4, SLC39A4, UBE3A, PDLIM3 and UBE21. If the patient has prostate cancer, the gene can be selected from the group consisting of BMP2, ENPEP, MCAM, SSBP2, PAK3, B4GALT1, and NDP. If the patient has lung cancer, the gene can be selected from the group consisting of PAK3, PIGH, TUBB4, B4GALT1, and NISCH. If the patient has breast cancer, the gene can be selected from the group consisting of KIF1A (kinesin family member 1A) and MAL (T cell proliferation protein). If the patient has colon cancer, the gene can be selected from the group consisting of GPR116, OSMR, PC4, SLC39A4, UBE3A, PDLIM3, NRTK2, OGDHL, SFRP4, SSBP2, and UBE21. If the cell is a cervical cancer cell, at least one gene can be selected from the group consisting of PDCD4, TFPI2, ARMC7, TRM-HUMAN, OGDHL, PTGS2, CDK6, GPR39, HMGN2, C130RF18, ASMTL, DLL4, NP-659450.1, NP-078820.1, CLU, HPCA, PLCG2, RALY, GNB4, CCNA1 NPTX1 and C9ORF19. Particularly the at least one gene can be selected from the group consisting of TFPI2, ARMC7, TRM_HUMAN, OGDHL, PTGS2, GPR39, C13ORF18, ASMTL, CCNA1, NPTX1 and DLL4.

According to additional aspects of the invention the finding of methylation of genes encoding proteins which are known to affect drug efficacy permits the use of methylation assays to predict response and stratify patients. For example, CBR-1 enhances the potency of doxorubicin, a chemotherapy drug. Methylation of the CBR-1 gene decreases the expression of CBR-1 thereby decreasing the potency of doxorubicin in the patient. Thus methylation of CBR-1 genes can be tested, and if found to be greater than in controls, than treatment with doxorubicin will be contraindicated. If methylation is not greater than in controls, such therapy is predicted to be efficacious. Similarly, methylation of genes such as TK-1, MYCK, and KCNJ8 can be used to predict drug efficacy and risk of disease. Methylation of TK-1 predicts a better response to DNA damaging agents, since TK-1 helps a cell circumvent the effects of DNA damaging agents. MYCK methylation can be used to predict the efficacy of methotrexate and mercaptopurinol treatment for leukemia. Similarly methylation of KCNJ8 can be used to predict risk of heart arrhythmia.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLE 1

### Methylation for Prostate Cancer

Data were collected during a re-expression experiment using the prostate cancer cell lines 22rv1, DU145, LNACAP, and PC3. Expression levels of cells treated with 5 μM 5-Azacytidine (DAC) were compared to identical cell lines not treated with this reagent by hybridization to an Affymetrix HGU133A chip using a standard protocol.

Analysis Strategy:

A. The datasets containing information on around 23.000 genes were copied from the data archive on the ‘Methalyzer’ to a newly created directory.

B. The needed details on the Affymetrix HGU133A chip were downloaded to the data analysis area of ‘Methalyzer’

C. The needed analysis tools (specific ‘R’ libraries for the bioconductor package) updated

D. To estimate the raw data quality, two graphical overviews were created:
Intensity plots for each chip on its own
RNA degradation plot

E. Data sets were normalized together using the tool called ‘expresso’ applying the following parameters:
Background correction: Mas
Normalization: quantiles
PM correction: Mas
Expression: Mas

F. The result of the normalization was tested using a boxplot which displayed the intensity level calculated for each gene present on the chip.

G. ‘P’ (present) ‘M’, (marginal) and ‘A’ (absent) calls made available by the MAS5 algorithm (Affymetrix software) for each gene and each experiment were collected and transferred to an Excel sheet.

H. Using MS Excel, ‘P’ and ‘A’ calls were converted into an ‘Expression Score’ using the following rules:
a. ‘P’ in the DAC treatment data sets got a score of 1
b. ‘A’ in the non-treatment data sets got a score of 1

I. For each gene, the Expression Score was calculated

J. A cut-off of 5 was defined as the first minimal criterion a gene had to fulfill

K. As certain genes are present more than once on the chips used, a Perl script was created which allowed linking the probe set name on the chip and the corresponding RefSeq ID.

L. The list of genes was restructured in such a way that each probe set was described in one row

M. The table was transferred into a purpose built MS Access database

N. A table containing the Methascores 2.2 of all described genes was added to the database system

O. The following filtering rules were applied to the dataset
a. X-chromosomal genes were excluded
b. Expression of the genes was ranked descending
c. Methascore 2.2 had to be >3 and the number of different patterns per gene had to be >3
   Reactivated genes are shown in Table 4.

### EXAMPLE 2

### Methylation for Lung Cancer

Data were collected during re-expression experiments using squamous lung cancer cell lines HTB-58 and HTB-59 as well as lung adenocarcinoma cell lines A549 and H23. Expression levels in cells treated with 2 μM 5-Azacytidine (DAC) were compared to identical cell lines not treated with this reagent (PBS as replacement) by hybridization to Affymetrix HGU133A chip and the HGU95av2 chip using a standard protocol.

Analysis Strategy:

A. The datasets containing information on the probe sets were copied from the data archive on the ‘Methalyzer’ to a newly created directory.

B. The needed details on the Affyrmetrix HGU133A chip and the HGU95av2 chip were downloaded to the data analysis area of ‘Methalyzer’

C. The needed analysis tools (specific ‘R’ libraries for the bioconductor package) updated

D. To estimate the raw data quality, two graphical overviews were created:
a. Boxplot of intensities for each chip before normalization
b. RNA degradation plot of each data set

E. Data sets were normalized together using the tool called ‘expresso’ applying the following parameters:
a. Background correction: Mas
b. Normalization: quantiles
c. PM correction: Mas
d. Expression: Mas

F. The result of the normalization was tested using a boxplot which displayed the intensity level calculated for each gene present on the chip.

G. ‘P’ (present) ‘M’, (marginal) and ‘A’ (absent) calls made available by the MAS5 algorithm (Affymetrix software) for each gene and each experiment were collected and transferred to an Excel sheet.

H. Using MS Excel, ‘P’ and ‘A’ calls were converted into an ‘Expression Score’ using the

following rules:

a. ‘P’ in the DAC treatment data sets got a score of 1

b. ‘A’ in the non-treatment data sets got a score of 1

I. For each gene, the Expression Score was calculated

J. A cut-off of 4 in the cans of squamous lung cancer cell lines and of 2 in the case of adenocarcinoma of the lung cell lines was defined as the first minimal criterion a gene had to fulfill

K. As certain genes are present more than once on the chips used, a Perl script was created which allowed to link the probe set name on the chip and the corresponding RefSeq ID.

L. The list of genes was restructured in such a way that each probe set was described in

one row

M. The table was transferred into a purpose built MS Access database

N. A table containing the Methascores 2.2 of all described genes was added to the database system

O. The following filtering rules were applied to the dataset

a. X-chromosomal genes were excluded

b. Expression scores were ranked descending and a cut-off of >4 (squamous) and >2 (adenocarcinoma of the lung) was set

c. Methascore 2.2 cut-off was set to >4 for both type of cell lines

Reactivated genes are shown in Tables 1, 2, and 3 for squamous lung cancers, adenocarcinoma lung cancers, and both lung cancers.

Summary analysis of both types of lung cancer cell lines:

This study consisted of a comparison of the results achieved with both types of cell lines. As two different chip generations with different technical specifications and only few common probe sets were used, the results were compared on a list by list basis. Both data sets have been normalized within chips and across chips in the right concept. Given this, it can be assumed that the results are valid on their own.

A comparison of the initial two lists indicated that there is one common element called “PLSC domain containing protein (LOC254531)”. This means that this marker can be used to detect both types of lung cancer but not to distinguish between both types of cancer. One additional marker for adenocarcinoma of the lung was found which doesn't occur in the squamous lung cancer cell line study (MCAM).

### EXAMPLE 3

### Methylation for Colorectal Cancer

Data were collected during a re-expression experiment using the colorectal cancer cell lines DLD-1, HCT116 and HT29. Expression of cells treated with 5 μM 5-Azacytidine (DAC) were compared to identical cell lines not treated with this reagent using a standard protocol and hybridization to Affymetrix HGU133A chips.

Analysis strategy:

A. The datasets containing information on around 23.000 genes were copied from the data archive on the ‘Methalyzer’ to a newly created directory.

B. The needed details on the Affymetrix HGU133A chip were downloaded to the data analysis area of ‘Methalyzer’

C. The needed analysis tools (specific ‘R’ libraries for the bioconductor package) updated

D. To estimate the raw data quality, two graphical overviews were created:
a. Intensity plots for each chip on its own
b. RNA degradation plot

E. Data sets were normalized together using the tool called ‘expresso’ applying the following parameters:
a. Background correction: Mas
b. Normalization: quantiles
c. PM correction: Mas
d. Expression: Mas

F. The result of the normalization was tested using a boxplot which displayed the intensity level calculated for each gene present on the chip.

G. ‘P’ (present) ‘M’, (marginal) and ‘A’ (absent) calls made available by the MAS5 algorithm (Affymetrix software) for each gene and each experiment were collected and transferred to an Excel sheet.

H. Using MS Excel, ‘P’ and ‘A’ calls were converted into an ‘Expression Score’ using the following rules:
a. ‘P’ in the DAC treatment data sets got a score of 1
b. ‘A’ in the non-treatment data sets got a score of 1

I. For each gene, the Expression Score was calculated

J. A cut-off of 4 was defined as the first minimal criterion a gene had to fulfill

K. As certain genes are present more than once on the chips used, a Perl script was created which allowed to link the probe set name on the chip and the corresponding RefSeq ID.

L. The list of genes was restructured in such a way that each probe set was described in one row

M. The table was transferred into a purpose built MS Access database

N. A table containing the Methascores 2.2 of all described genes was added to the database system

O. The following filtering rules were applied to the dataset
a. X-chromosomal genes were excluded
b. Expression of the genes was ranked descending
c. Methascore 2.2 had to be >3 and the number of different patterns per gene had to be >3

Reactivated genes are shown in Table 7.

### EXAMPLE 4

### Methylation for Cervix Cancer

Data were collected during re-expression experiments of the four cell lines Hela, Siha, CSCC7 and CSCC8. The cell lines were treated with three different concentrations of 5-Azacytidine (DAC; 0.2 μM, 1 μM, 5 μM) using otherwise identical experimental conditions.

Analysis strategy:

A. The datasets containing information on around 54.000 genes were copied from the data archive on the ‘Methalyzer’ to a newly created directory.

B. The needed details on the Affymetrix HGU133Aplus2.0 chip were downloaded to the data analysis area of ‘Methalyzer’

C. The needed analysis tools (specific ‘R’ libraries for the bioconductor package) updated

D. To estimate the raw data quality, two graphical overviews were created:
a. Intensity plots for each chip on its own
b. RNA degradation plot

E. Data sets were normalized together using the tool called ‘expresso’ applying the following parameters:
a. Background correction: Mas
b. Normalization: quantiles
c. PM correction: Mas
d. Expression: Mas

F. The result of the normalization was tested using a boxplot which displayed the intensity level calculated for each gene present on the chip.

G. ‘P’ (present) ‘M’, (marginal) and ‘A’ (absent) calls made available by the MAS5 algorithm (Affymetrix software) for each gene and each experiment were collected and transferred to an Excel sheet.

H. Using MS Excel, ‘P’ and ‘A’ calls were converted into an ‘Expression Score’ using the following rules:
a. ‘P’ in the DAC treatment data sets got a score of 1
b. ‘A’ in the non-treatment data sets got a score of 1

I. For each gene and condition, the Expression Score was calculated

J. Lists were sorted based on a minimal expression score which was identical to the number of chips available for each condition

K. The Methascore cut-off was set to >3 in all data sets

L. Lists were created detailing the conditions used and the markers selected

M. A summary was created which contained a condensed representation of the findings including an overview on which markers occurred under which condition.

Reactivated genes are shown in Table 8.

### EXAMPLE 5

### Methylation for Breast Cancer

Data were collected during a re-expression experiment using the breast cancer cell lines BT-20, MCF-7, MDA-MB 231 and MDA-MB 436. Expression levels of cells treated with 5 μM 5-Azacytidine (DAC; in acetic acid; were compared to identical cell lines not treated with this reagent (PBS as replacement) using an Affymetrix HGU133A chip.

Analysis strategy:

A. The datasets containing information on around 23.000 genes were copied from the data archive on the ‘Methalyzer’ to a newly created directory.

B. The needed details on the Affymetrix HGU133A chip were downloaded to the data analysis area of ‘Methalyzer’

C. The needed analysis tools (specific ‘R’ libraries for the bioconductor package) updated

D. To estimate the raw data quality, two graphical overviews were created:
a. Intensity plots for each chip on its own
b. RNA degradation plot

E. Data sets were normalized together using the tool called ‘expresso’ applying the following parameters:
a. Background correction: Mas
b. Normalization: quantiles
c. PM correction: Mas
d. Expression: Mas

F. The result of the normalization was tested using a boxplot which displayed the intensity level calculated for each gene present on the chip.

G. ‘P’ (present) ‘M’, (marginal) and ‘A’ (absent) calls made available by the MAS5 algorithm (Affymetrix software) for each gene and each experiment were collected and transferred to an Excel sheet.

H. Using MS Excel, ‘P’ and ‘A’ calls were converted into an ‘Expression Score’ using the following rules:
a. ‘P’ in the DAC treatment data sets got a score of 1
b. ‘A’ in the non-treatment data sets got a score of 1

I. For each gene, the Expression Score was calculated

J. A cut-off of 4 was defined as the first minimal criterion a gene had to fulfill

K. As certain genes are present more than once on the chips used, a Perl script was created which allowed to link the probe set name on the chip and the corresponding RefSeq ID.

L. The list of genes was restructured in such a way that each probe set was described in one row

M. The table was transferred into a purpose built MS Access database

N. A table containing the Methascores 2.2 of all described genes was added to the database system

O. The following filtering rules were applied to the dataset
a. X-chromosomal genes were excluded
b. Expression scores were ranked descending and a cut-off of >4 was set
c. Methascore 2.2 cut-off was set to >3

Reactivated genes identified for breast are shown in Table 6.

### EXAMPLE 6

### Cervical cancer

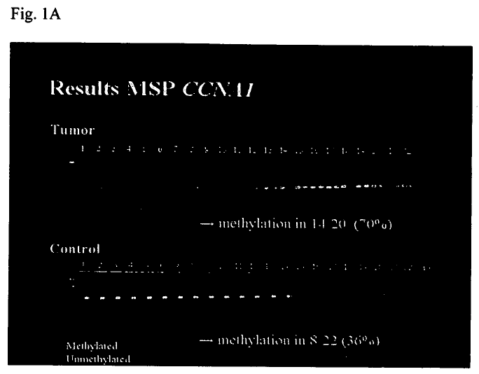

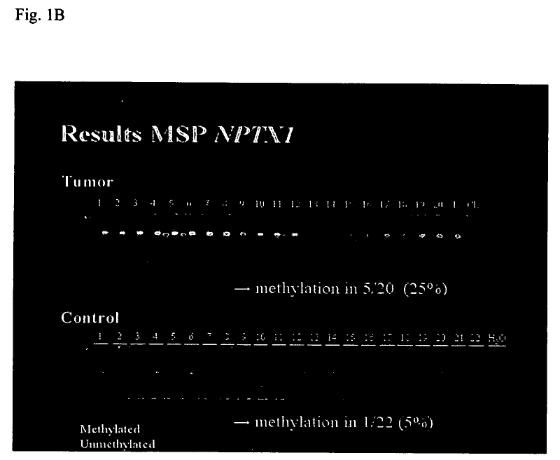

Three markers (CCNA1, NPTX1 and CACNA1C) were analyzed with Methylation Specific PCR in patient samples:
CCNA1 and NPTX1 discriminate between cancers and normal cervixes (see FIG. 1).
CACNA1C→inadequate marker (methylated in cancers as well as in normal cervixes)

For the other markers direct bisulfite sequencing (BSP) was performed on DNA derived from cervix samples from subjects without (normals) and with cervical cancer. Until now one (1) of the 24 tested markers showed methylation in the DNA from normals, 12 were unmethylated, 3 were almost completely unmethylated with the exception of one CG site and 8 were mostly unmethylated but showed methylation in more than 1 CG site.

For 12 markers BSP results are available in cancer tissues: 10 of these contain methylated cytosines. The markers TFPI2, ARMC7, TRM_HUMAN, OGDHL, PTGS2, GPR39, C13ORF18, ASMTL and DLL4 show differential methylation between the normals and the cancers cases.

### EXAMPLE 7

### Prostate Cancer

The methylation status of 47 genes was considered in the prostate cancer cell lines 22rv1; DU145; LNACAP and PC3. Markers CDH1, PTGS2, TWIST1, EDNRB, RUNX3, RARB, FANCF, FHIT and NMU have been reported previously to be methylated in prostate tissue or other tissue types. GLDC, RPS28, PODXL, ARIH2, ANAPC2, ARMC8, CSTF2T, POLA, FLJ10983, ZNF398, CBLL1, HSPB6, NF1, CEBPD, ARL4A, ARTS-1, ETFDH, PGEA1, HPN and WDR45 were found to be unmethylated in prostate cell lines.

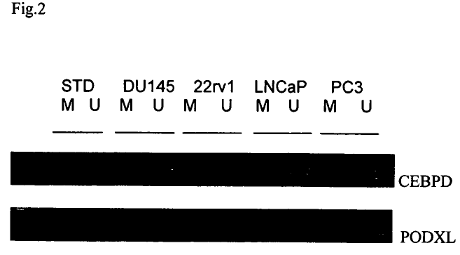

Sixteen out of 47 genes were shown to be methylated in at least some of the prostate cell lines by way of direct bisulfite sequencing. Genes NDP, CD3D, APOC1, NBL1, MCAM, ING4, LEF1, CENTD3, MGC15396 were methylated in all four cancer cell lines. FKBP4 was methylated in cell lines 22rv1, LNCaP and PC3; PLTP was methylated in cell lines 22rv1, LNCaP and PC3; genes ATXN1 and TFAP2A were methylated in cell lines DU145 and LNCaP; ENPEP was methylated in cell lines DU145 and PC3; SSBP2 was methylated in cell lines LNCaP and PC3 and gene BMP2 was methylated in cell line DU145. For other markers the methylation status was tested by way of MSP. FIG. 2A visualizes the result obtained for the CEBPC and PODXL genes in the different cell lines by way of MSP.

The methylation status of the 16 genes was further tested in primary human prostate tissue and compared to their methylation status in normal prostate tissue from a non-prostate cancer patient. The markers BMP2, ENPEP, MCAM, SSBP2 and NDP show differential methylation between the normal prostate tissues and prostate cancer tissue or/and benign prostate hyperplasia.

### EXAMPLE 8

### Lung Cancer

The methylation status of 30 genes was considered in 15 lung adenoma-carcinoma/cancer cell lines by way of direct bisulfite sequencing or MSP. The Methprimer primer program was used to position the CpG island on the input sequence and to design primers.

A total of 18 out of the 30 genes appeared to be unmethylated at the first CPG island (BS1) in the cell lines tested, whereas twelve out of 30 genes were methylated at BS1 in at least some of the lung cell lines. No CpG islands could be identified in the FMO4 gene

Genes found to be unmethylated at BS1 were tested for their methylation status at subsequent CpG islands. One further gene, NISCH which was unmethylated at BS1 was found to be methylated at a further CpG island.

The genes evidenced to be be methylated in the tested cell lines, were further tested on methylation in 12 tumors and compared to 6 non-lung cancer patients. Table 18 indicates that markers PAK3, PIGH, TUBB4 and NISCH show differential methylation between the normals and the cancer cases.

### EXAMPLE 9

### Breast Cancer

Direct bisulfite sequencing or MSP was performed on DNA derived from different breast cancer cell lines (M12, BT20, M7, 231, 436 and HS578T). Genes BACH1, CKMT, GALE, HMG20B, KRT14, OGDHL, PON2, SESN1, KIF1A (kinesin family member 1A) and MAL (T cell proliferation protein) were methylated.

Among the listed genes in table 19, those indicated by gray were found to be unmethylated in the tested cell lines, or they were methylated breast cancer cell lines as well as in primary paired normals. Genes indicated by blancs are left to finish sequencing in paired normal and tumor tissues.

Genes which are non-methylated in nornals, but which are methylated in breast cancer tissue can be used for identifying or prognosis of breast cancer. In particular methylation markers for breast cancer are KIF1A (kinesin family member 1A), MAL (T cell proliferation protein).

### EXAMPLE 10

### Colon Cancer

Direct bisulfite sequencing or MSP was performed on DNA derived from colon cancer cell lines.

Bisulfite-sequencing. Bisulfite-modified genomic DNA was amplified by PCR using 10× buffer (166 mM (NH₄)₂SO₄, 670 mM Tris Buffer (pH 8.8), 67 mM MgCl₂, 0.7% 2-mercaptoethanol, 1% DMSO), cervix, and primer sets that were designed to recognize DNA alterations after bisulfite treatment. Primer sequences are shown in Table in the PPT file; PCR reaction was performed for 45 cycles of 96° C. for 1 min, 54° C. for 1 min, and 72° C. for 1 min. PCR products were gel-extracted (Qiagen, Valencia, Calif.) and sequenced using the ABI BigDye cycle sequencing kit (Applied Biosystems, Foster City, Calif.).

Conventional methylation-specific PCR (C-MSP). Bisulfite-treated DNA was amplified with either methylation-specific or unmethylation-specific primer sets by PCR using 10× buffer (166 mM (NH₄)₂SO₄, 670 mM Tris Buffer (pH 8.8), 67 mM MgCl₂, 0.7% 2-mercaptoethanol, 1% DMSO) supplemented with 1.5 μl of 50 mM MgSO₄ for RGL-1, 1 μl of 50 mM MgSO₄ for B4GAL1 and BAG-1. PCR reaction was performed for 35 cycles of 95° C. for 30 sec, 59° C. for 30 sec, and 72° C. for 30 sec in 25 μl of reaction volume.

All of the 36 genes B4GALT1, C10orf119, C10orf13, CBR1, COPS4, COVA1, CSRP1, DARS, DNAJC10, FKBP14, FN3KRP, GANAB, HUS1, KLF11, MRPL4, MYLK, NELF, NETO2, PAPSS2, RBMS2, RHOB, SECTM1, SIRT2, SIRT7, SLC35D1, SLC9A3R1, TTRAP, TUBG2, FLJ20277, MYBL2, GPRI16, OSMR, PC4, SLC39A4, UBE3A, PDLIM3 and UBE21 appeared to be methylated in the colon cancer cell lines. The methylation status of the DNAJC10 gene was not uniform.

23 out of 26 genes were successfully sequenced in primary colon cancer tissues (Table 20). Differential methylation markers for colon cancer are GPR116, OSMR, PC4, SLC39A4, UBE23 and UBE21. Among 36 genes, 13 genes are left to finish sequencing in paired normal and tumor tissues.

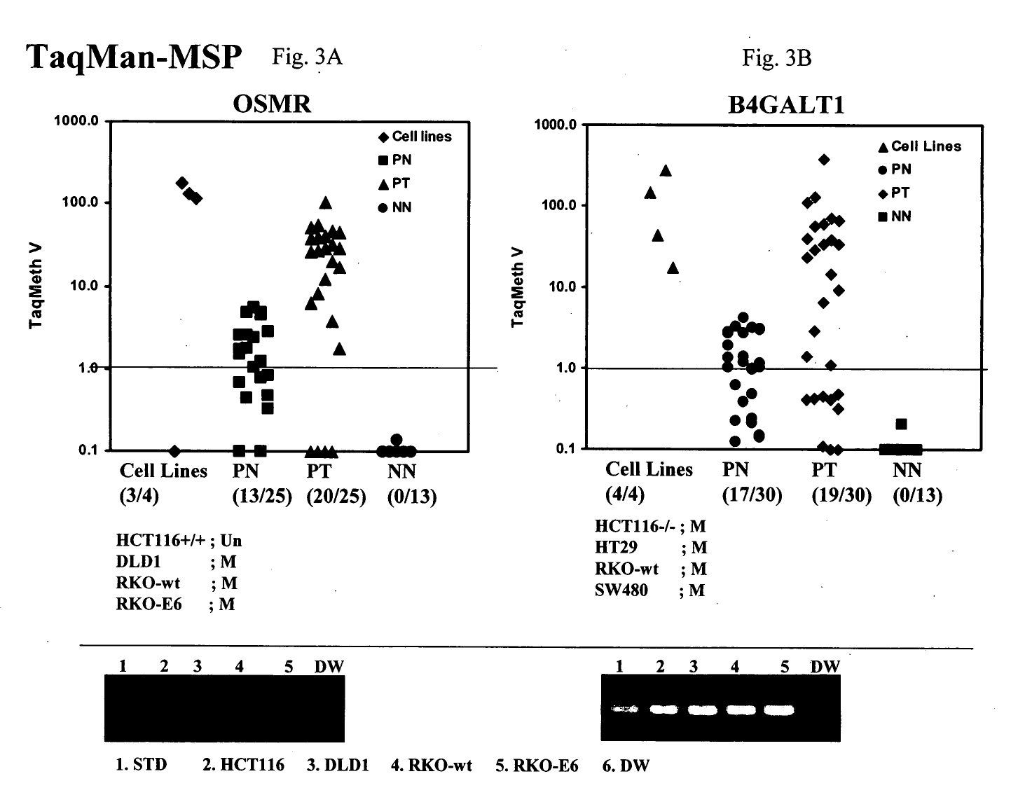

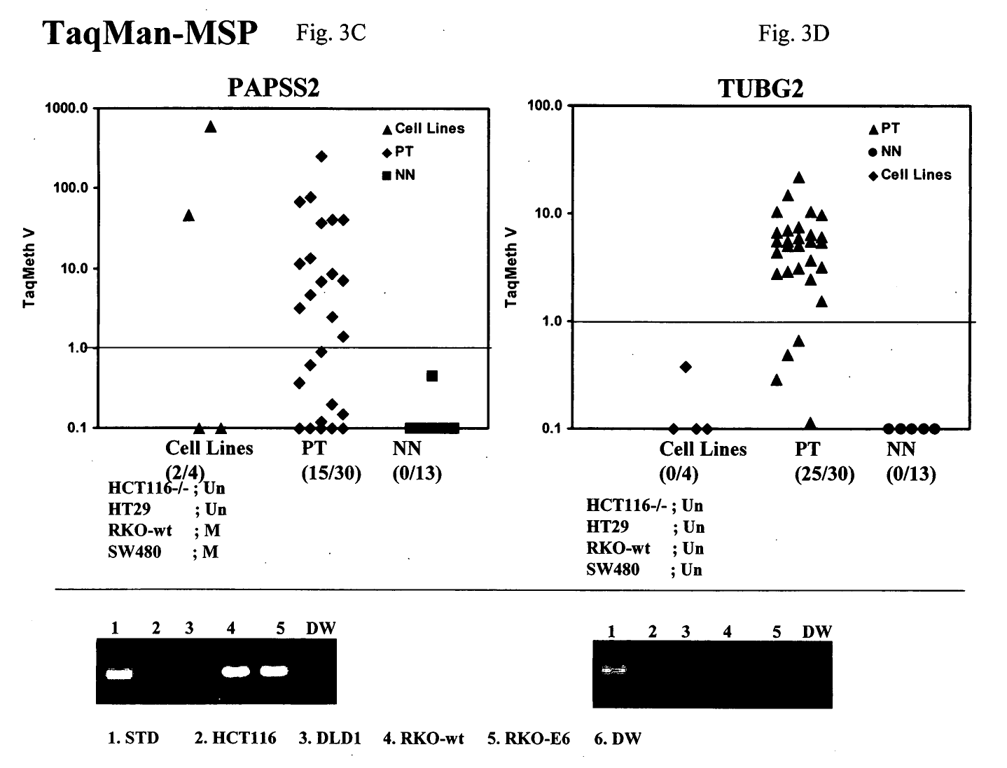

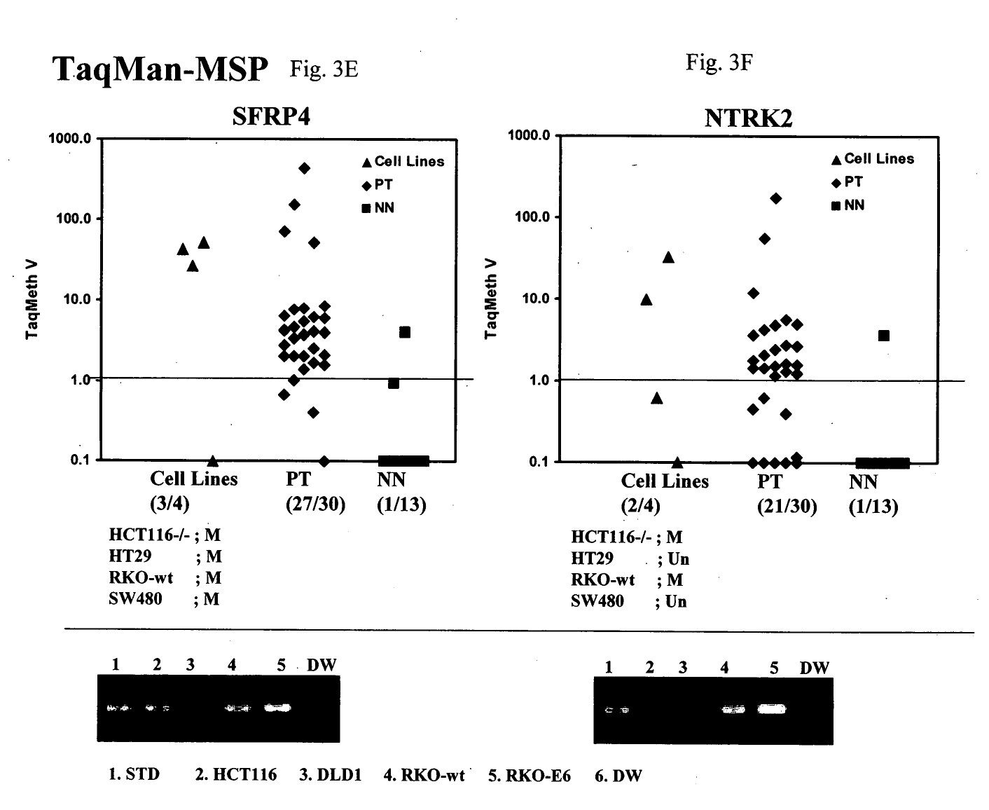

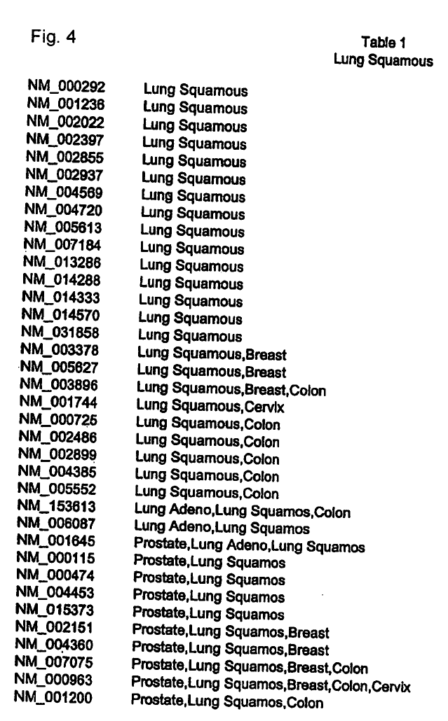

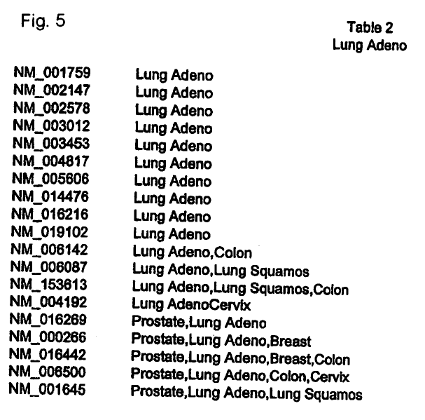

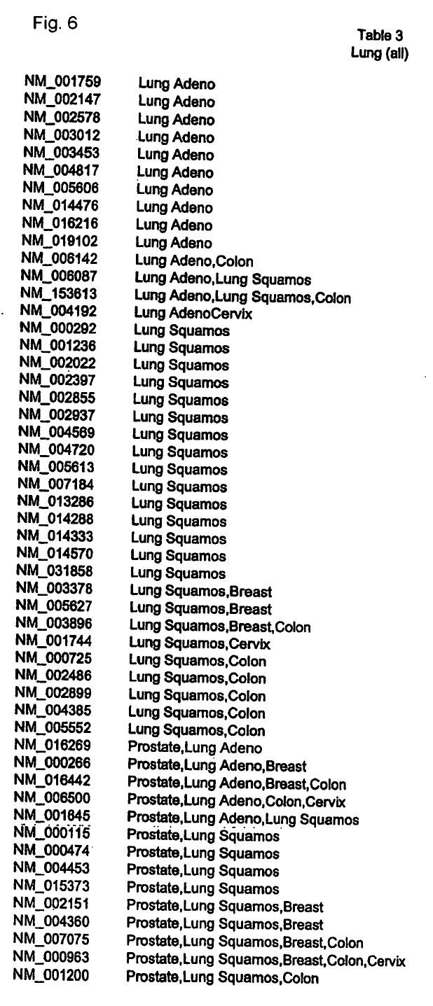

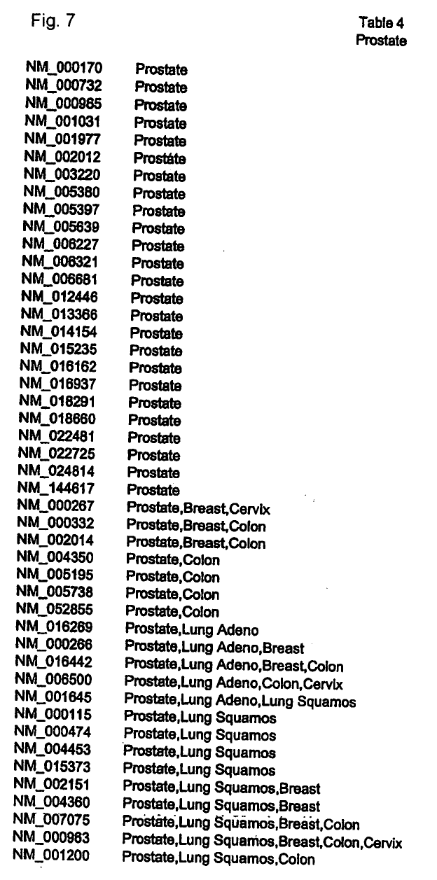

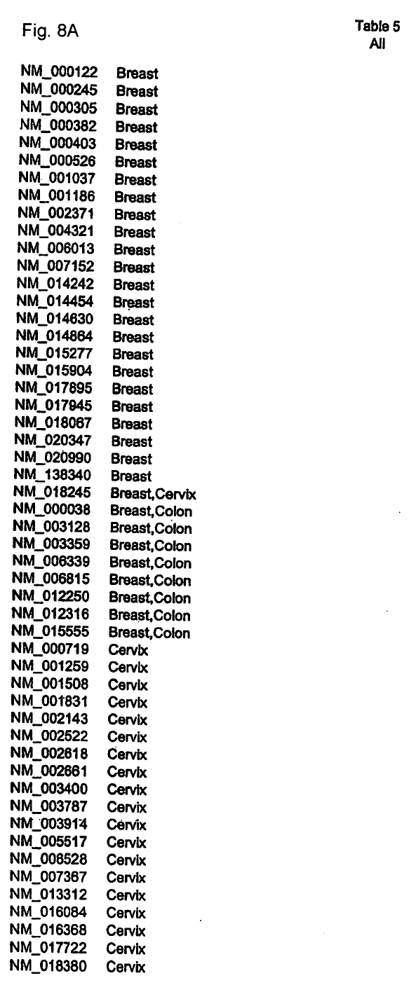

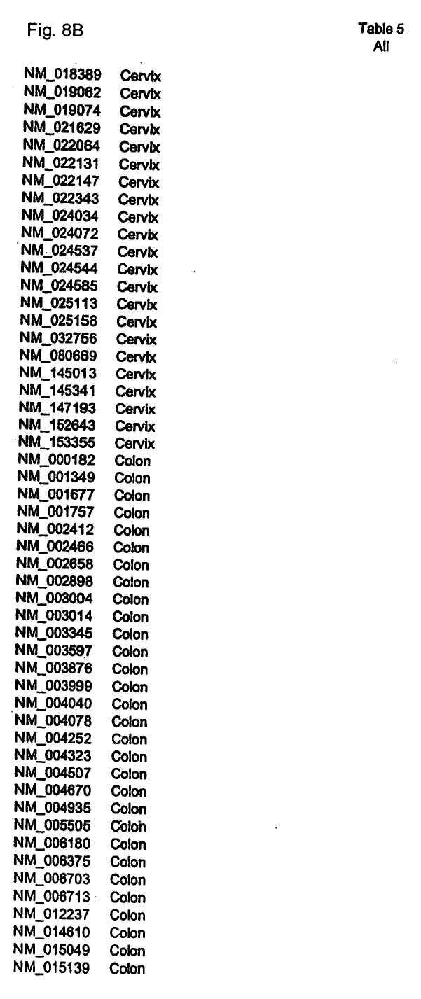

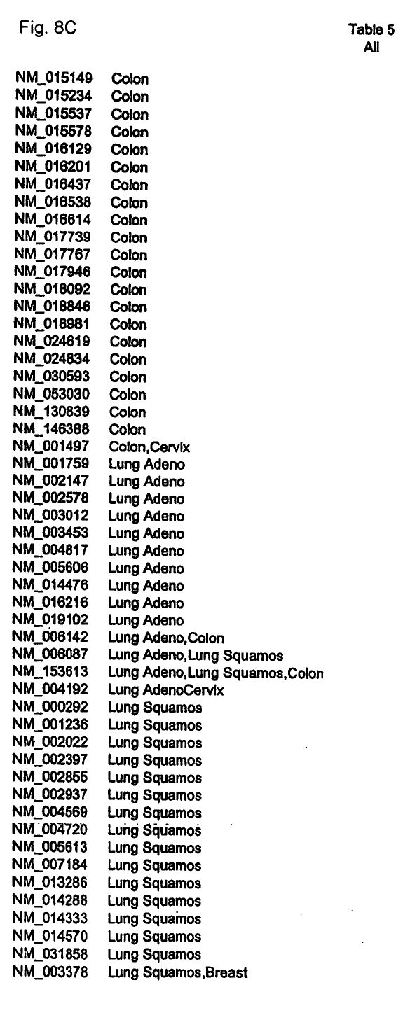

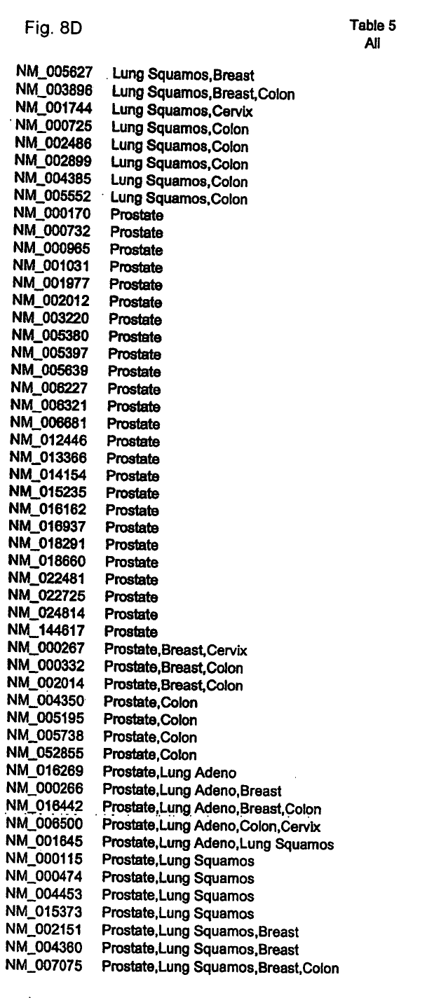

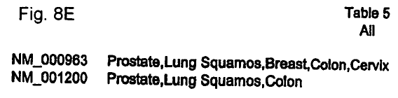

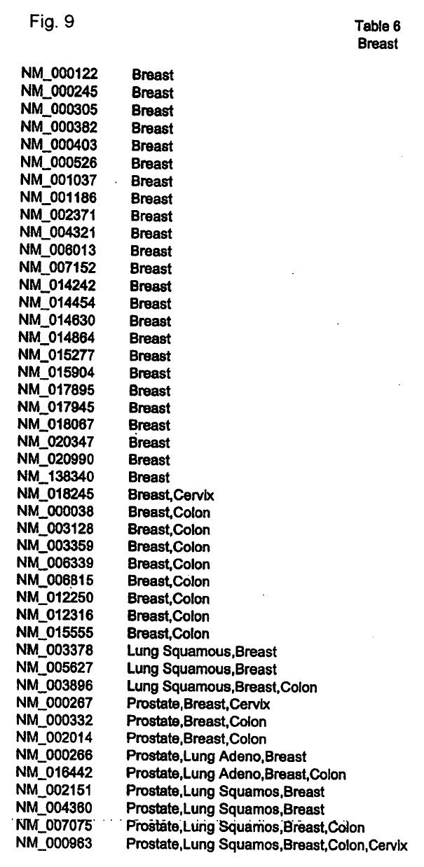

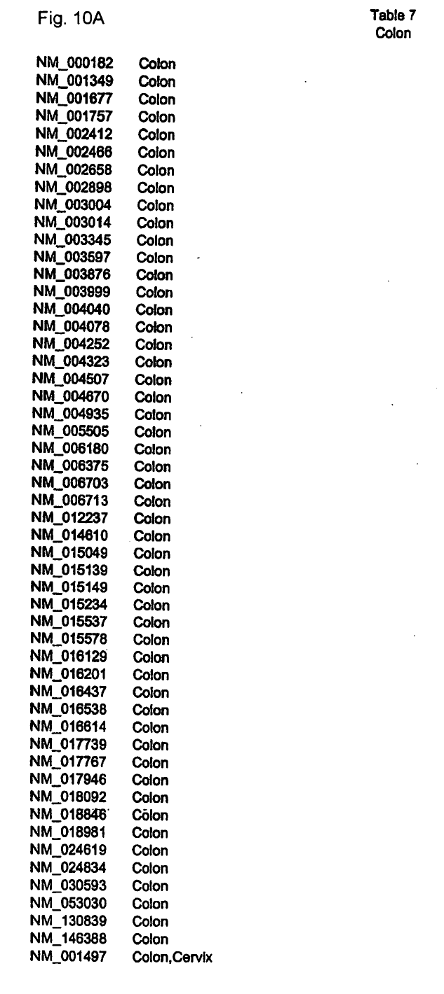

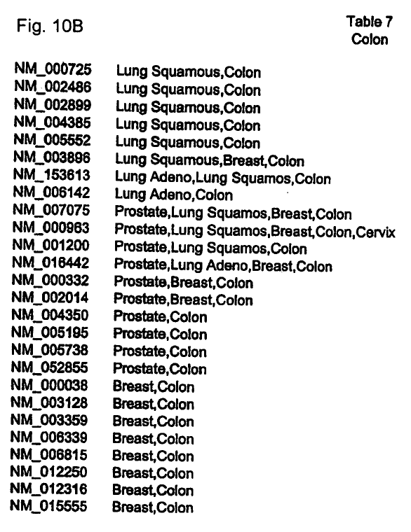

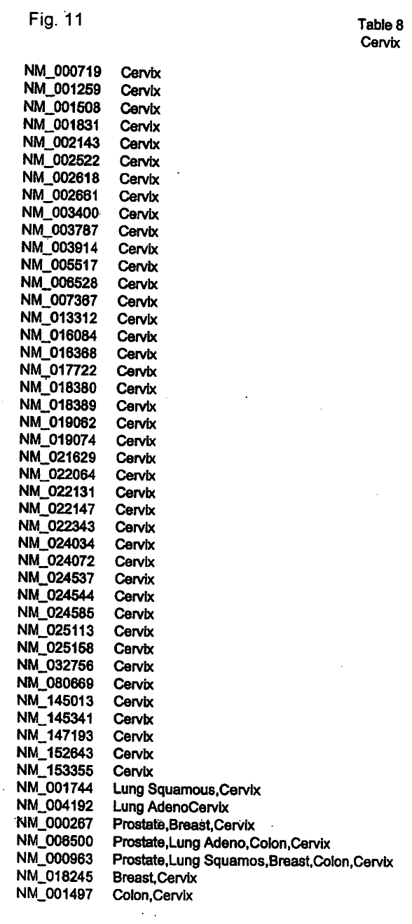

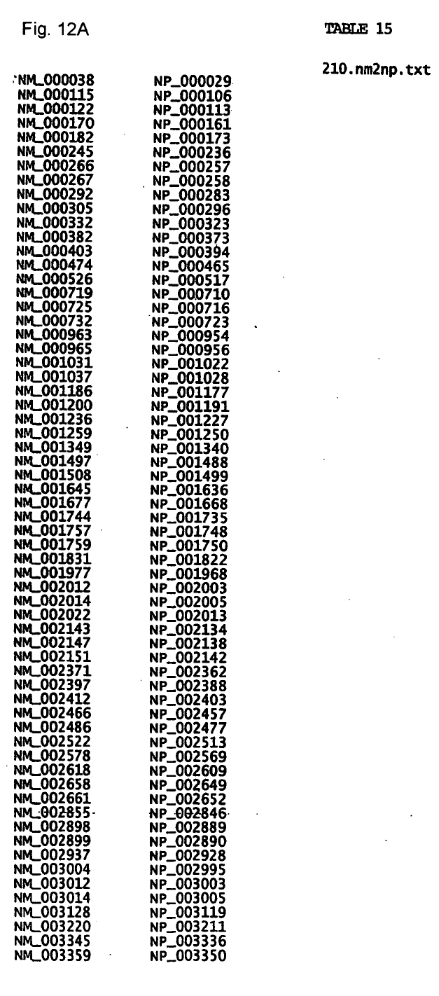

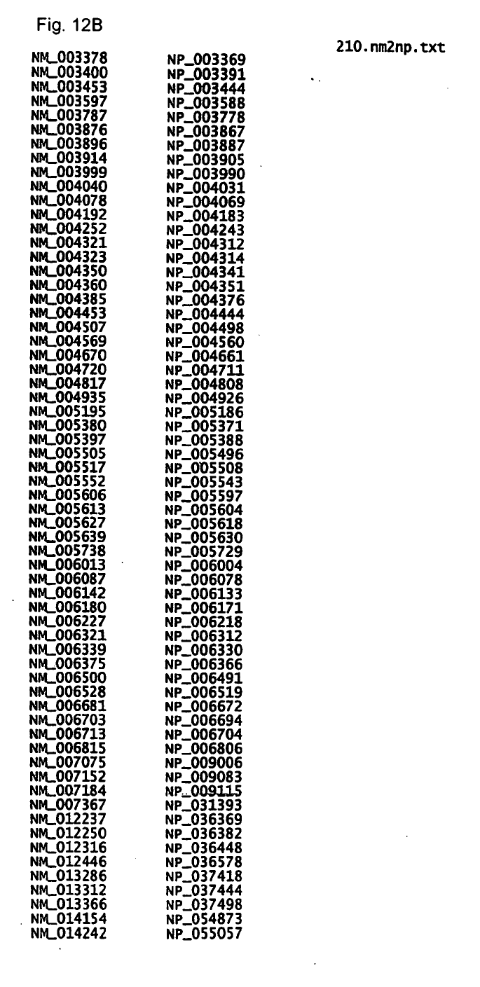

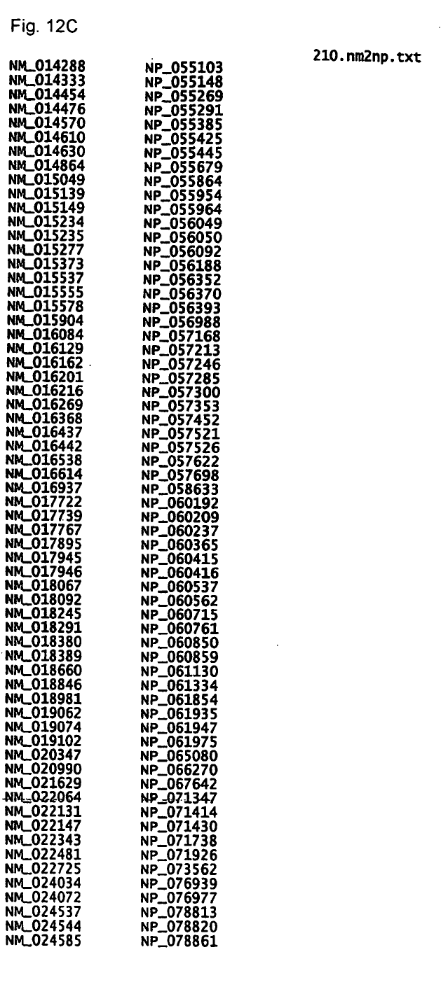

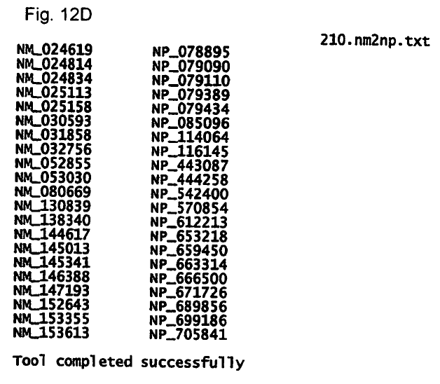

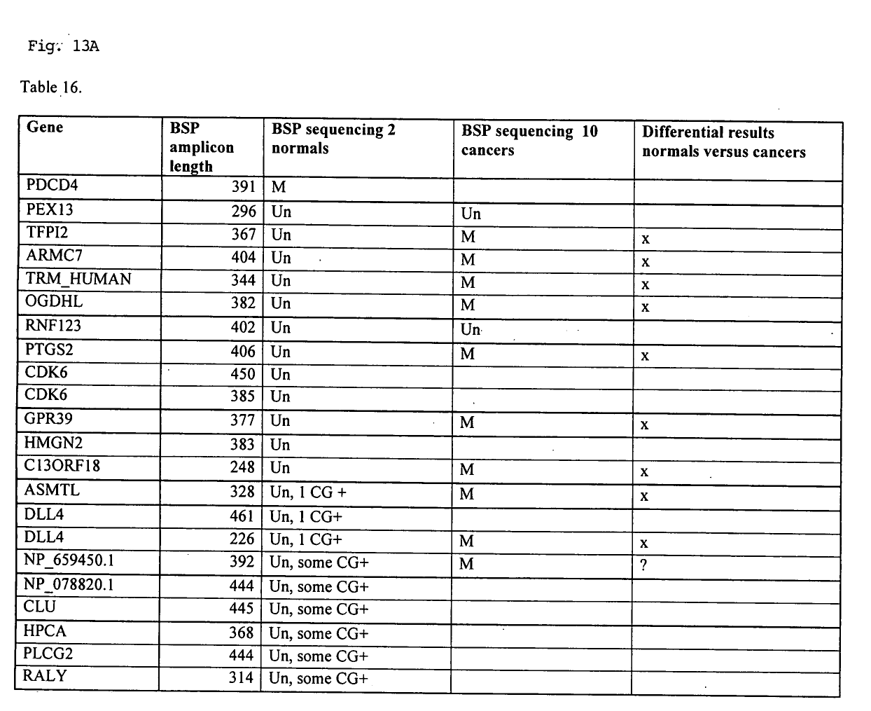

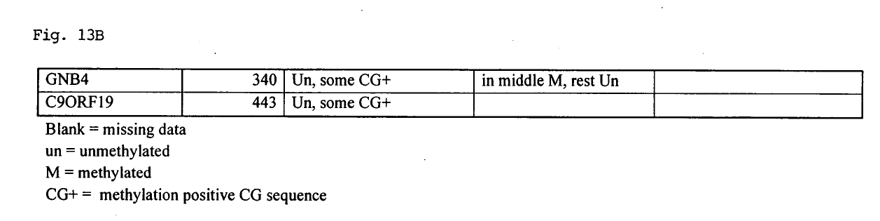

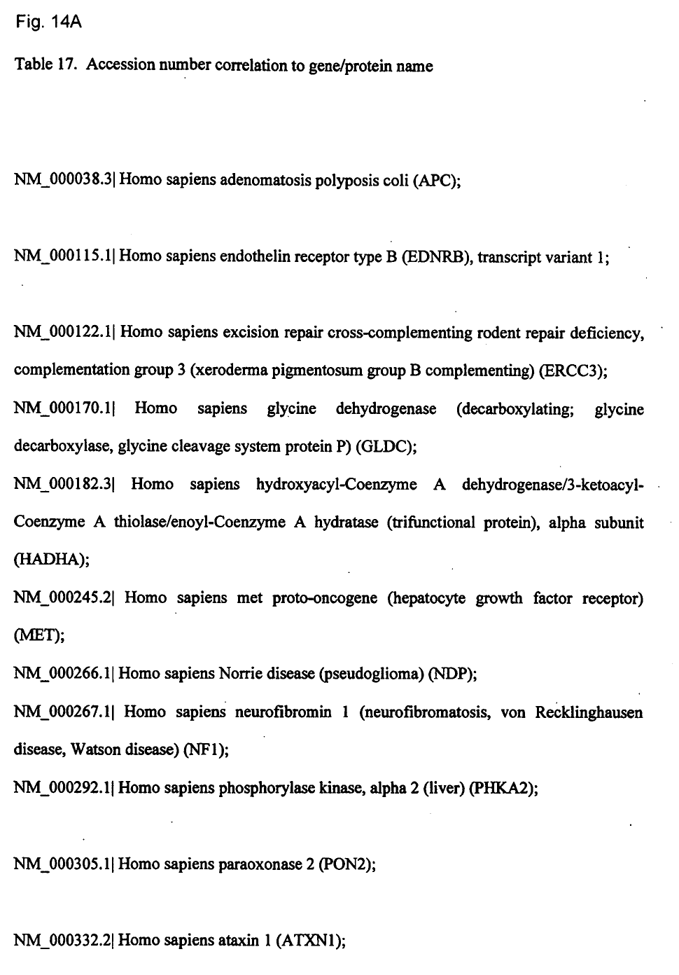

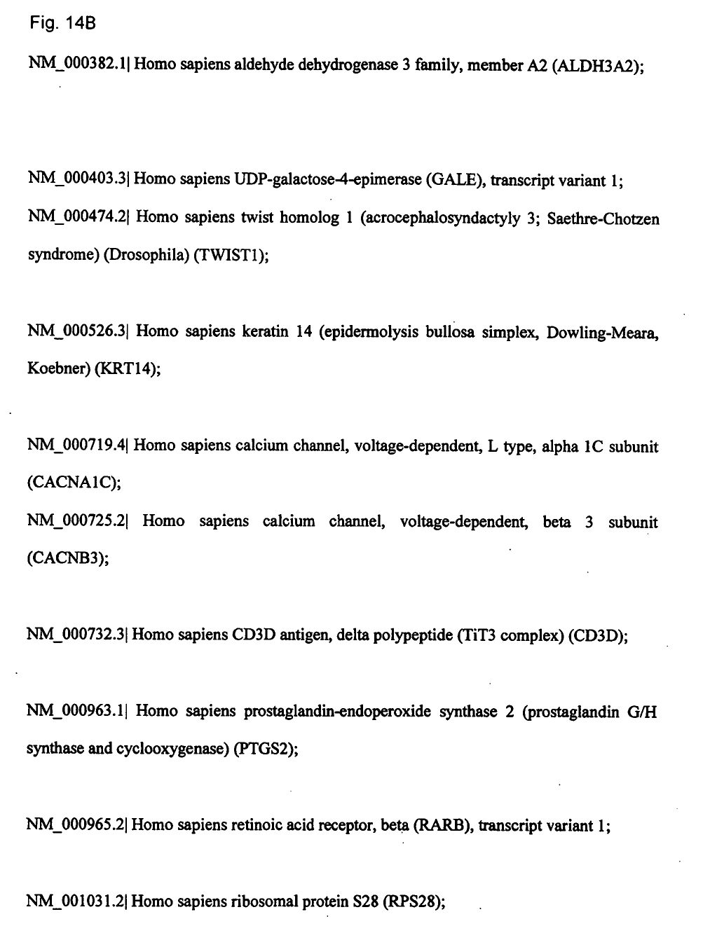

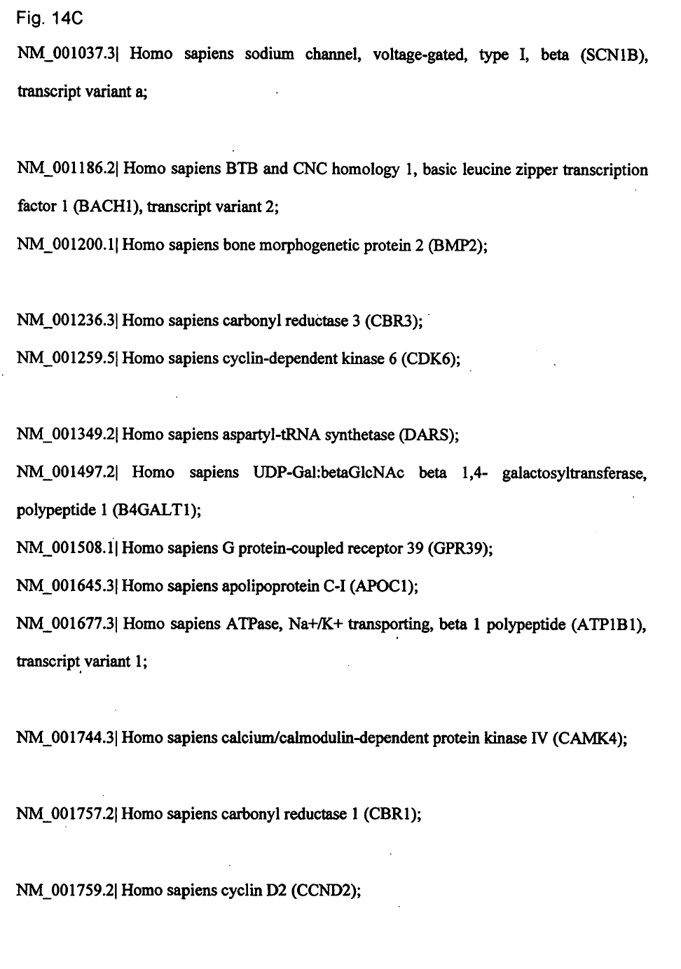

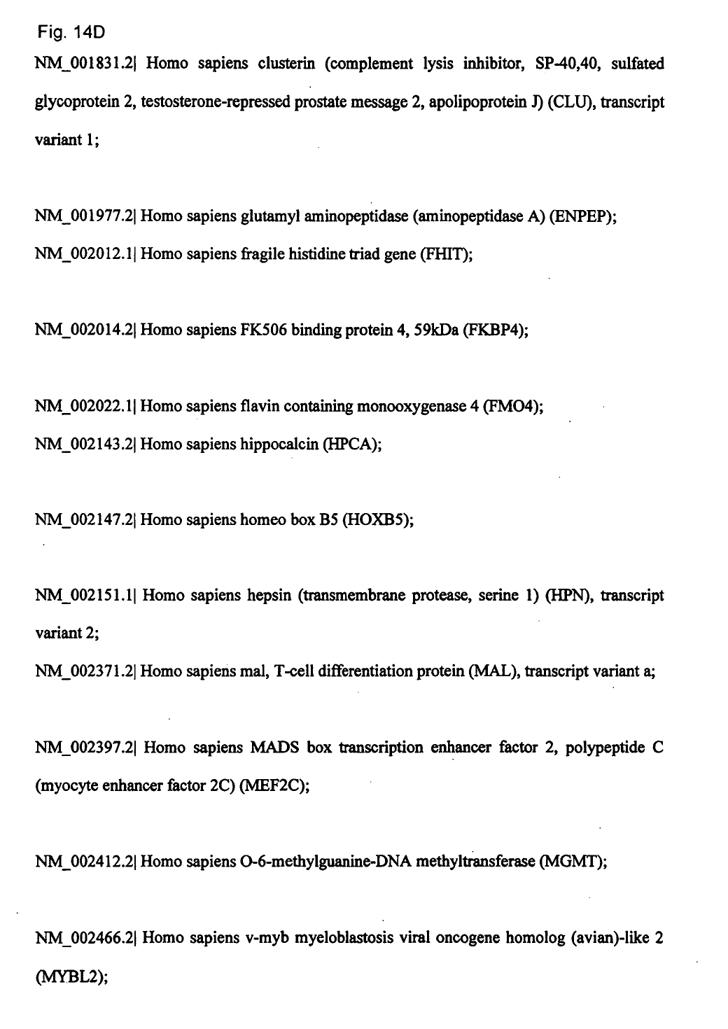

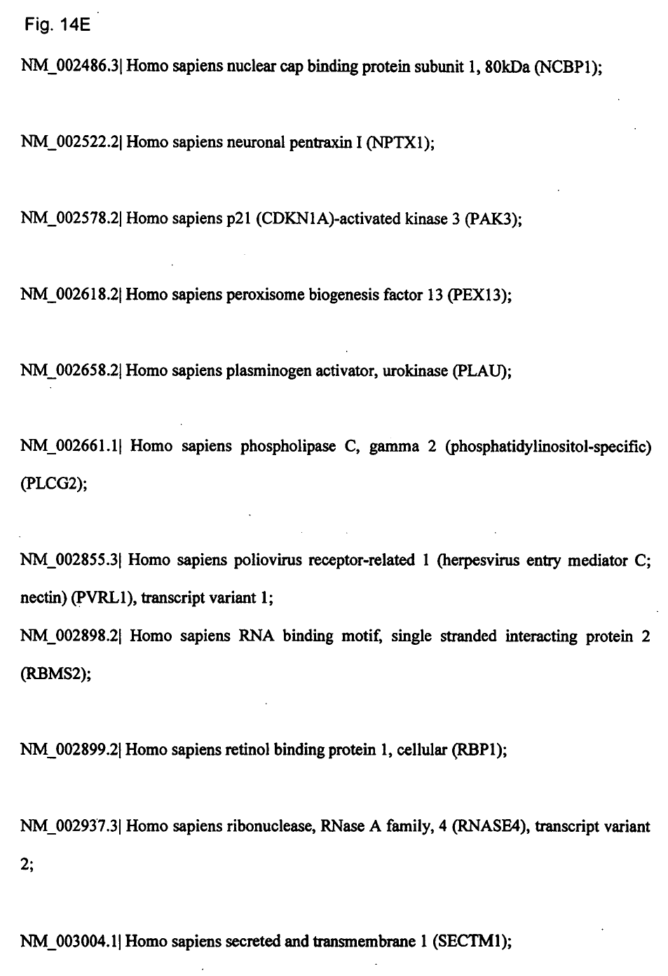

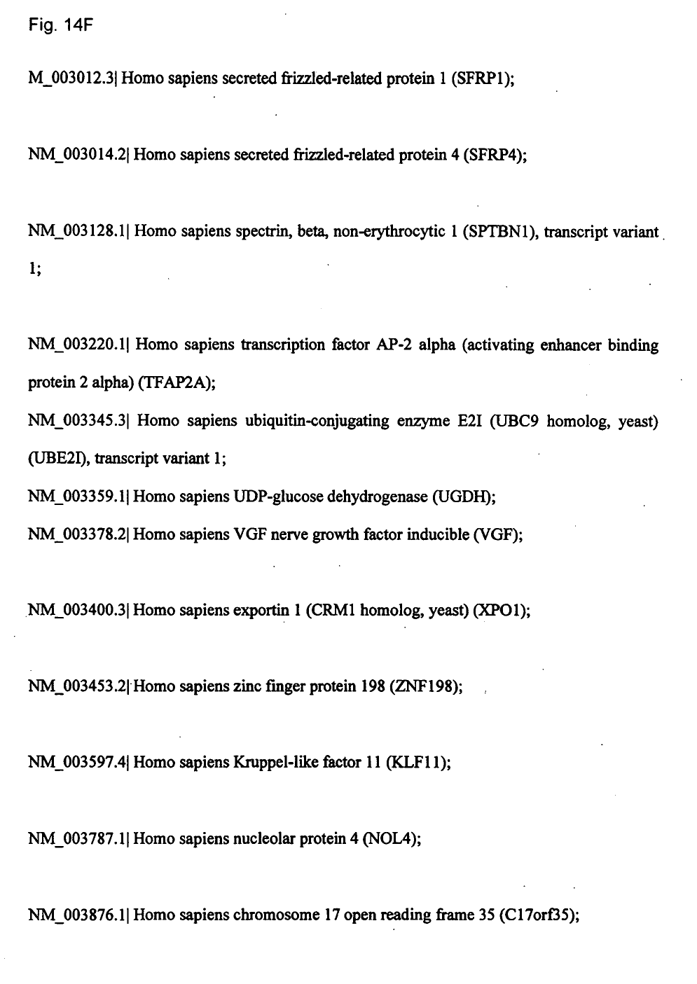

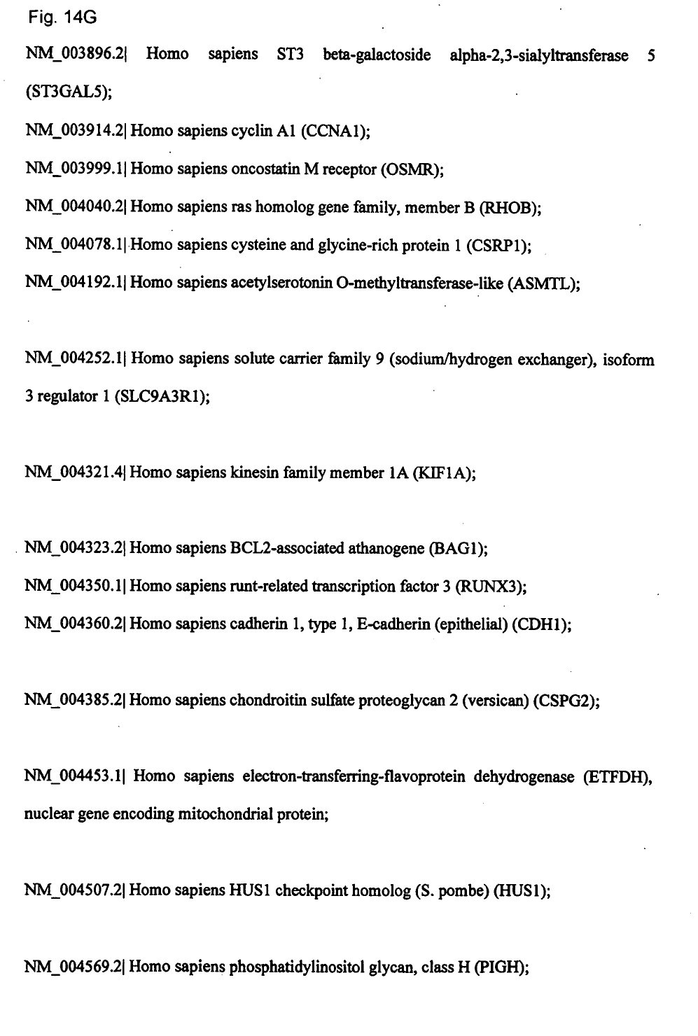

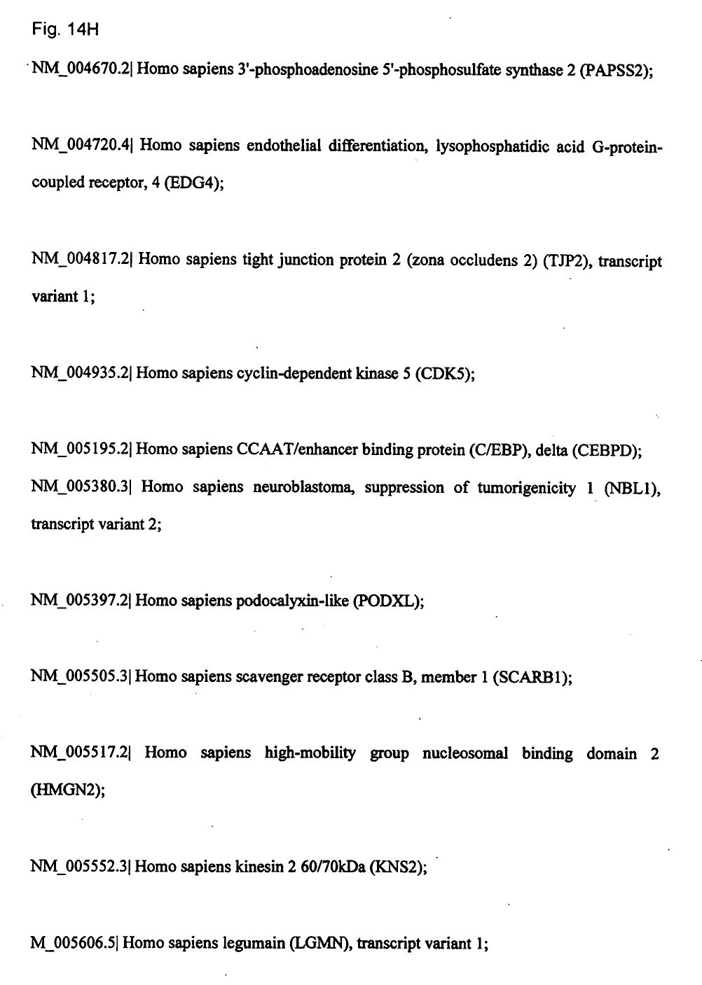

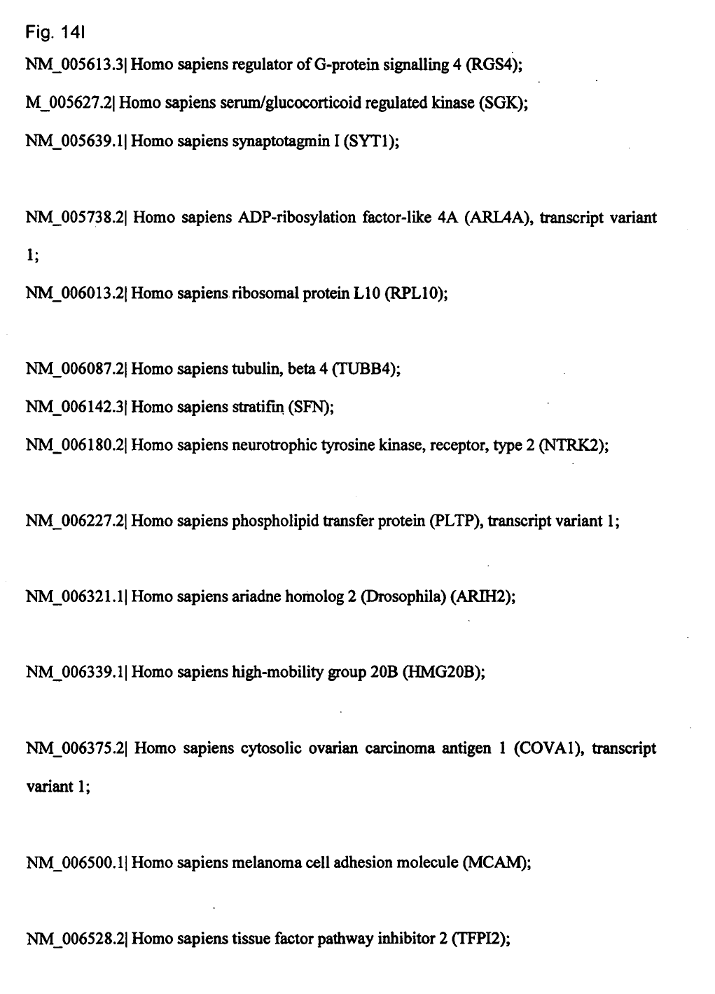

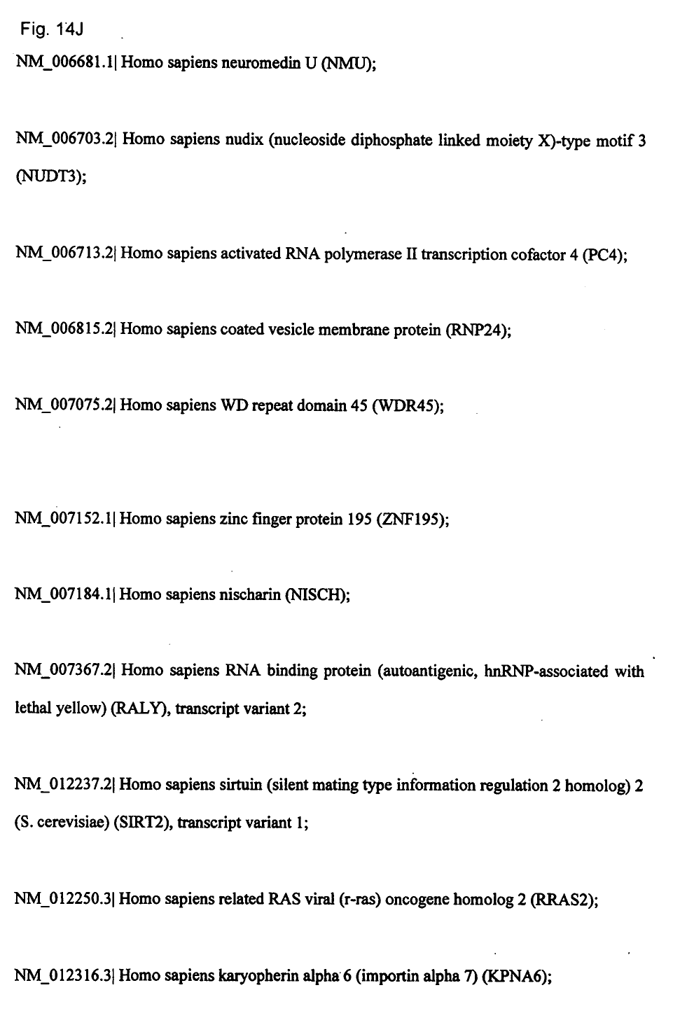

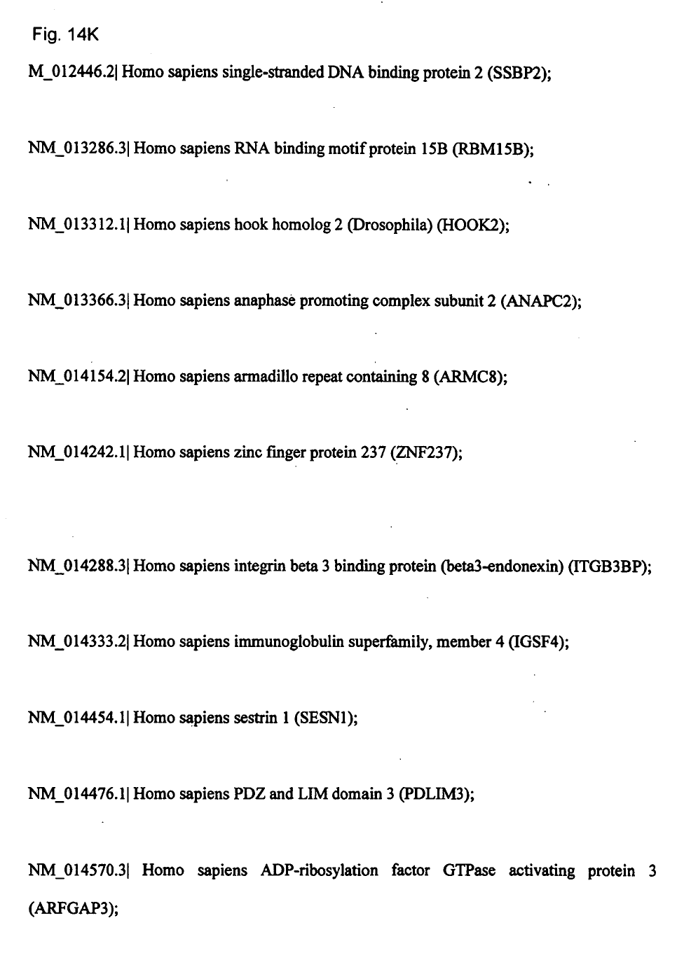

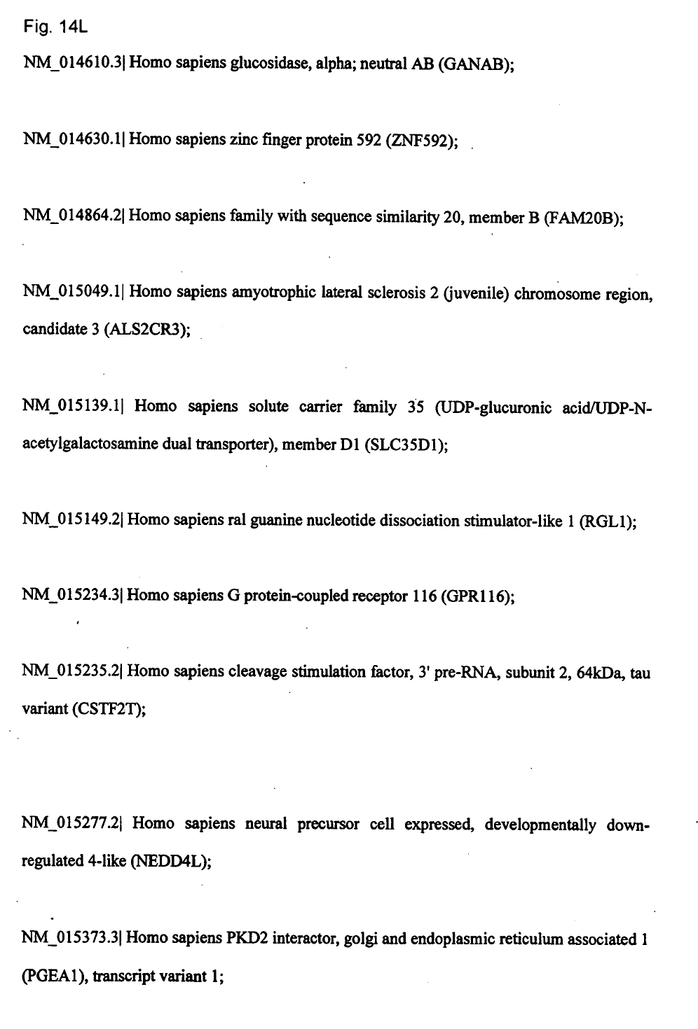

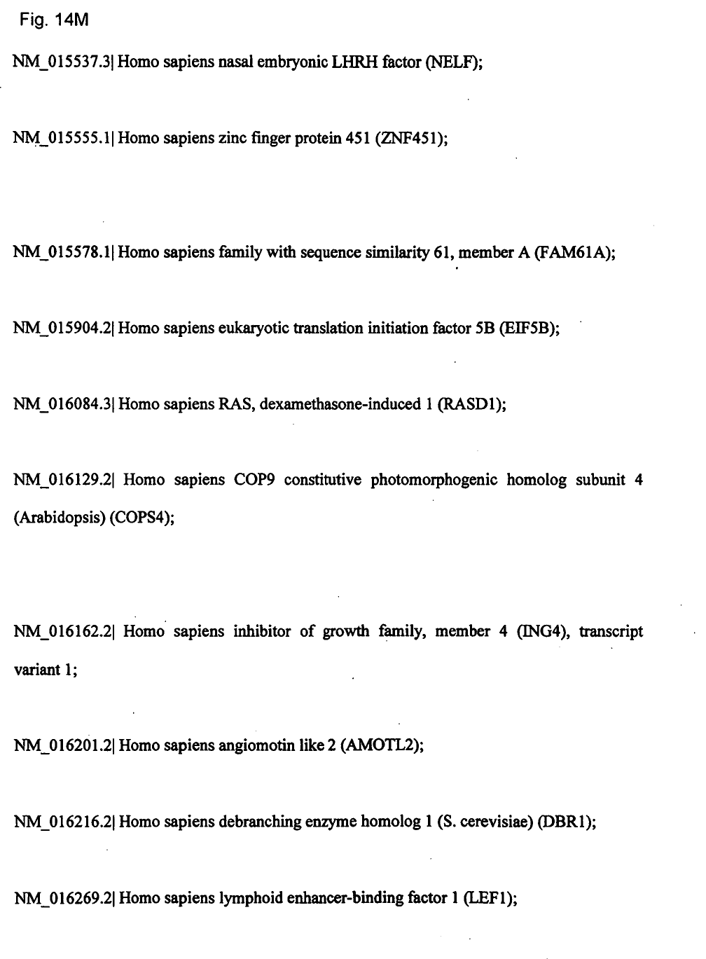

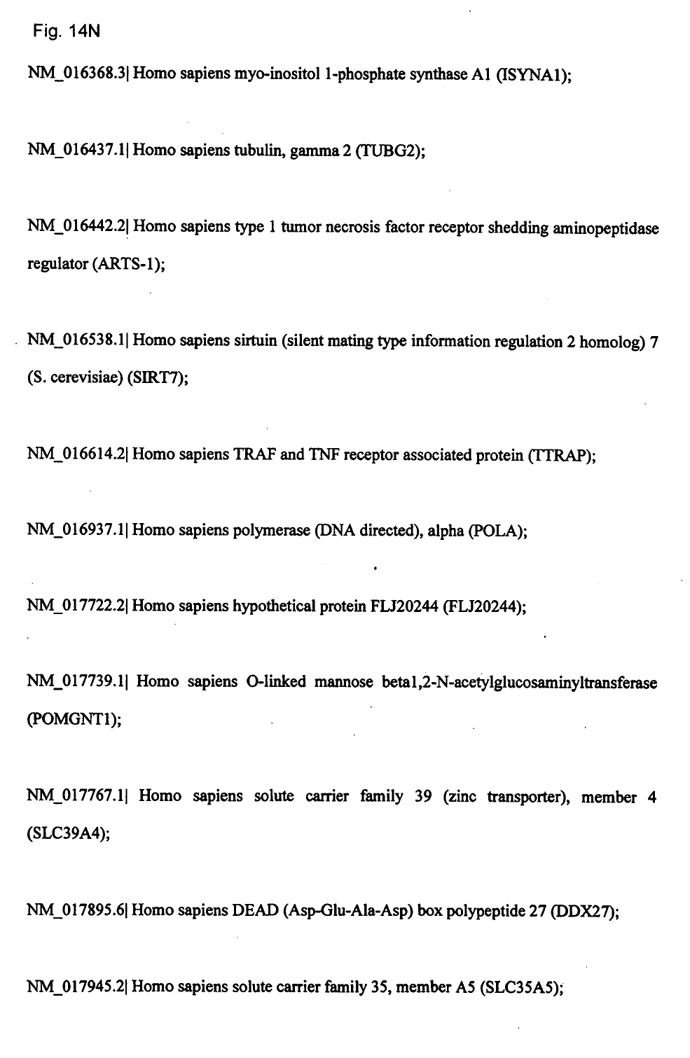

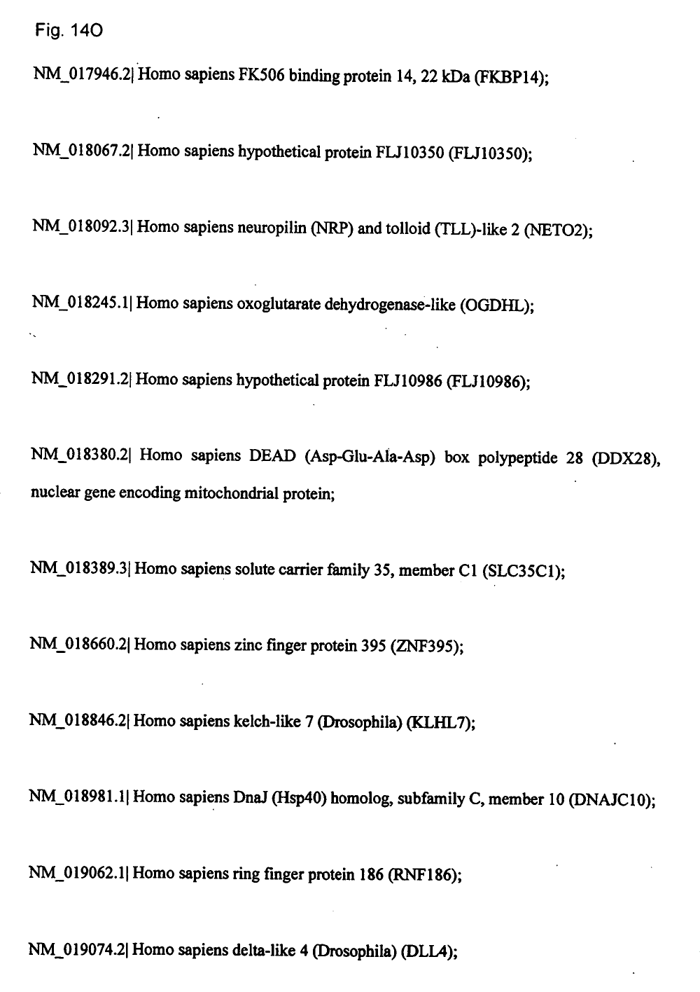

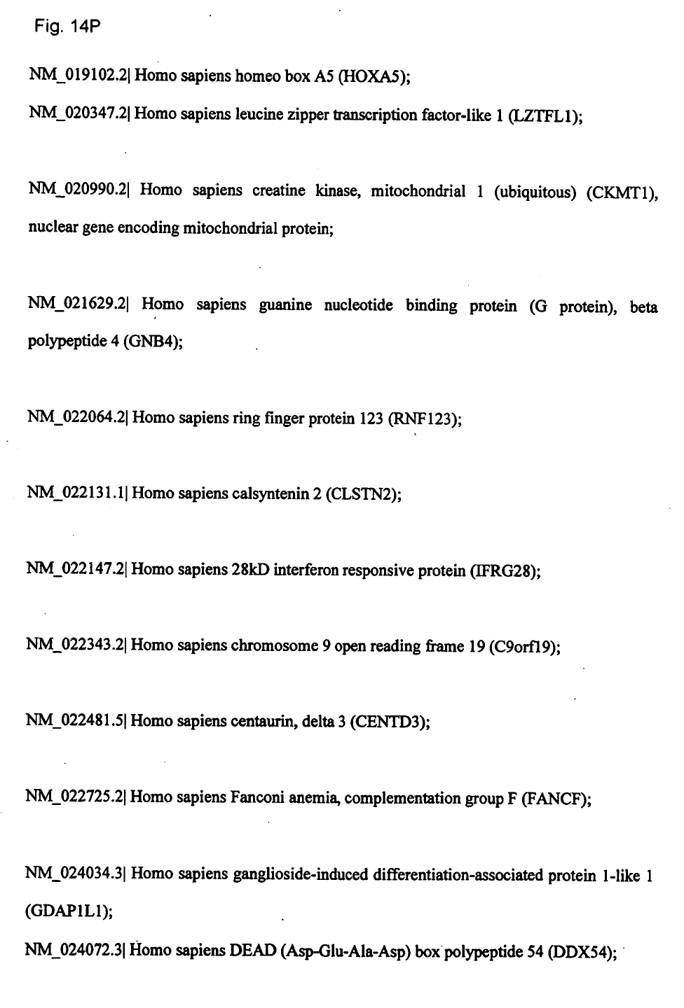

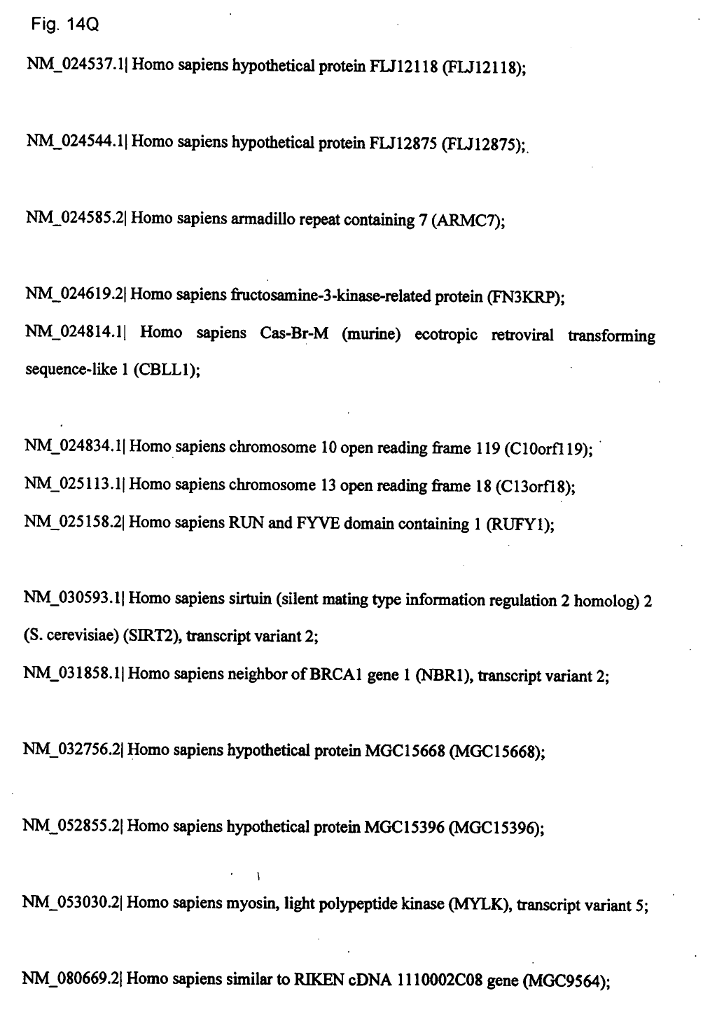

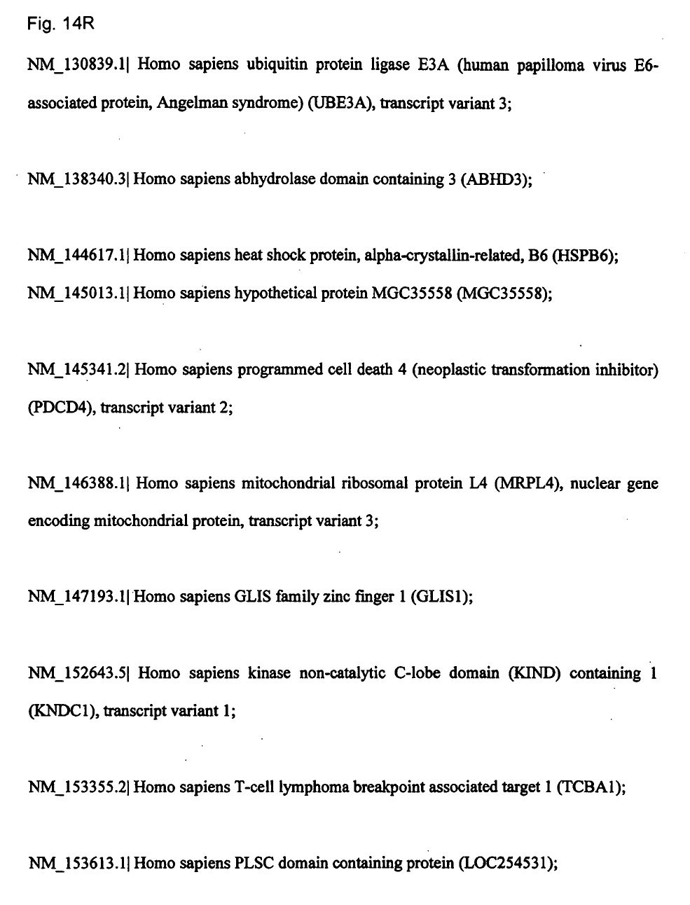

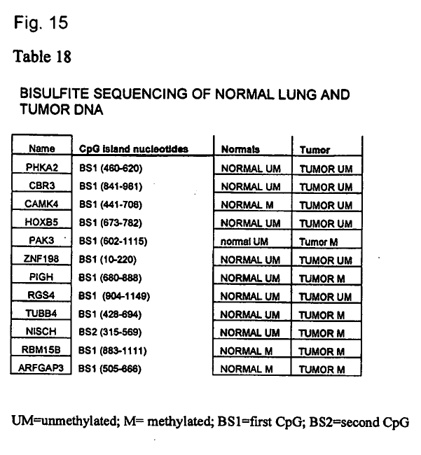

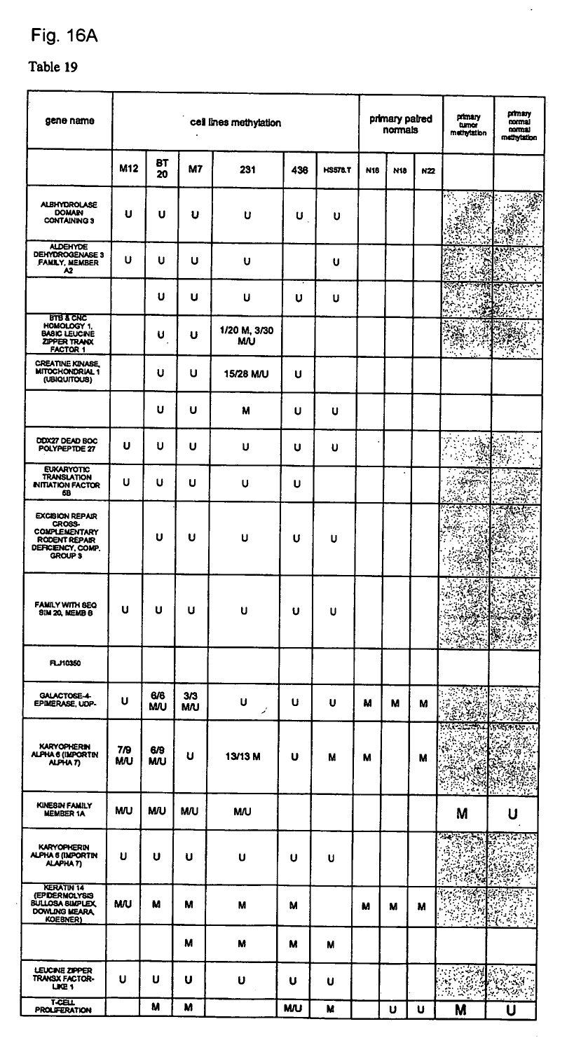

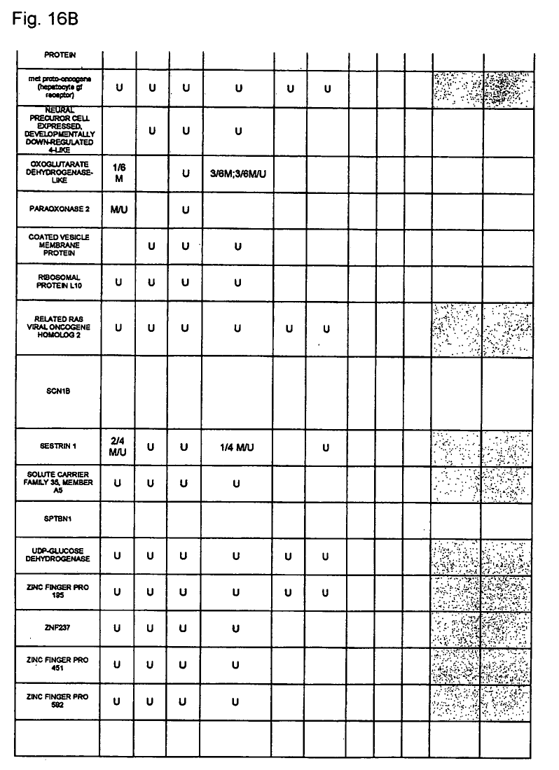

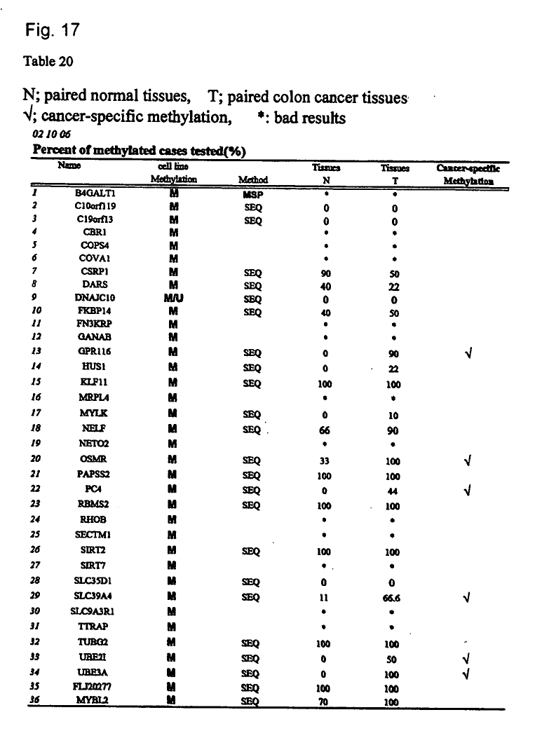

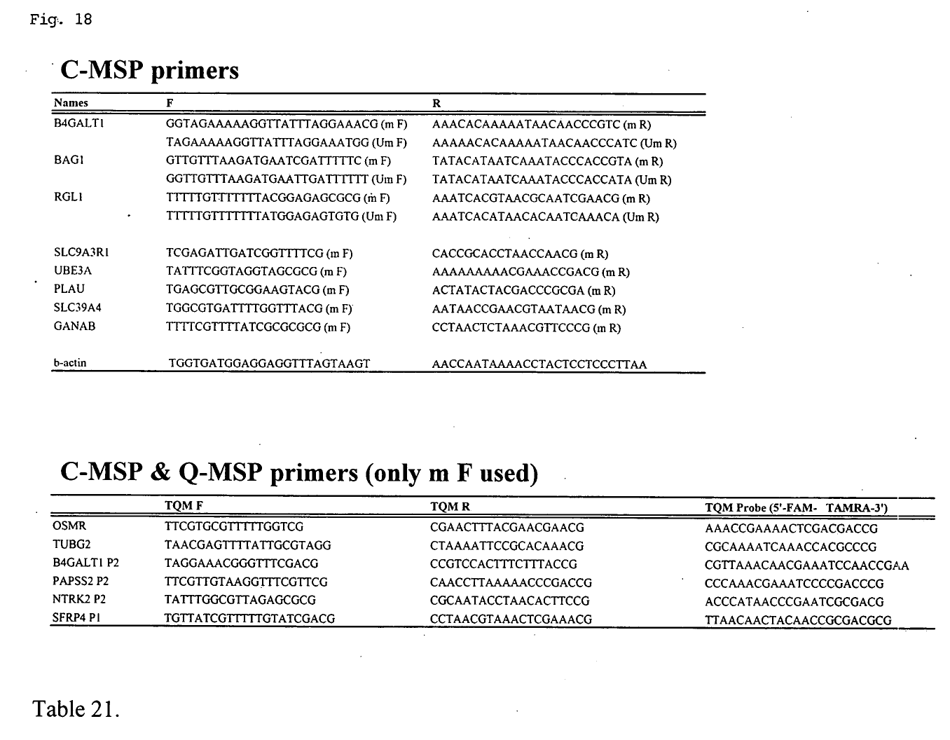

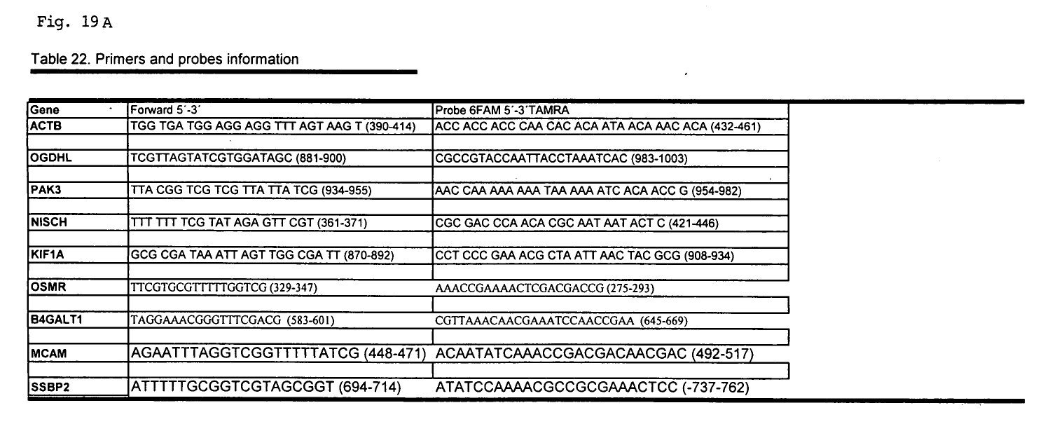

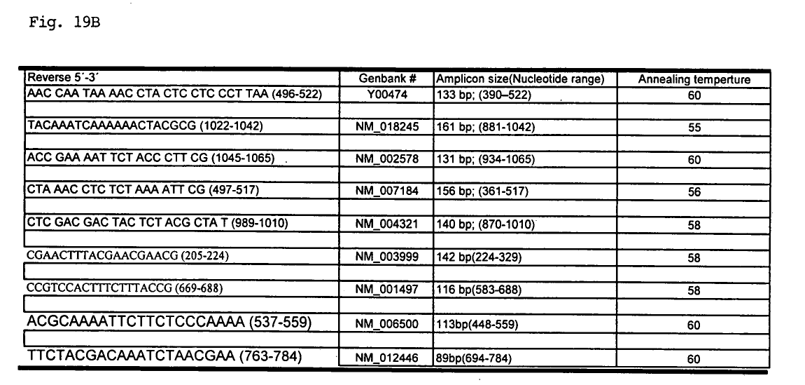

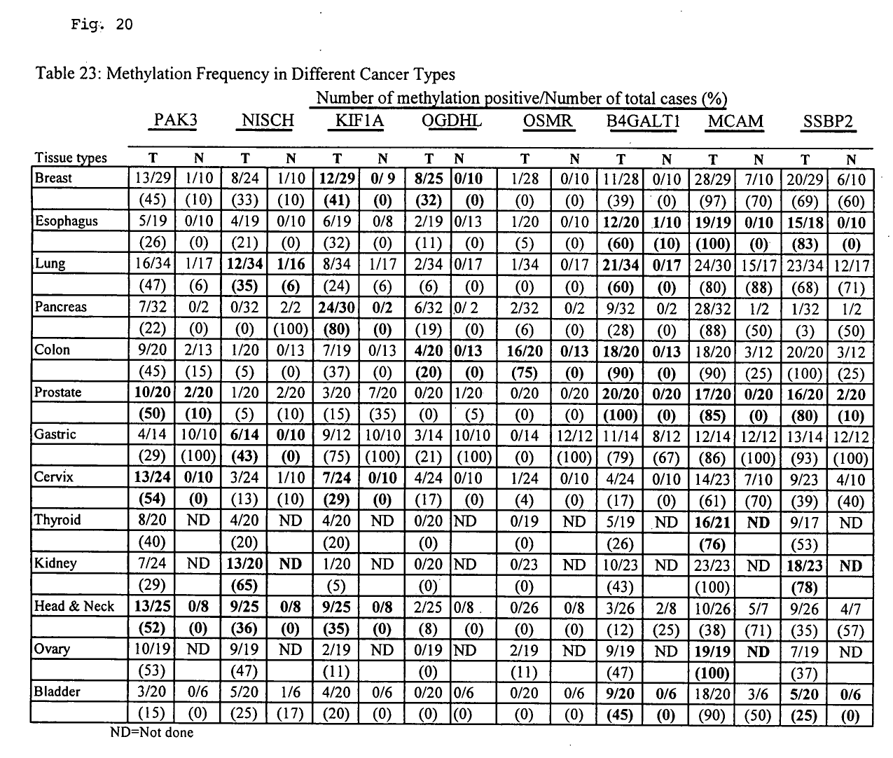

Further CMSP and QMSP assays were performed on tissue samples with primers shown in Table 21. Tissue tested were paired normals (PN), paired colorectal cancers (PT), normal epithelial components (NN); inflammatory cells, stromal cells and normal colon epithelial cells. Results are shown in FIG. 3A-3F and differential methylation was observed in tissue for B4GALT1, OSMR, PAPSS2, TUBG2, NTRK2 and SFRP4.

### EXAMPLE 10

### Various Cancers

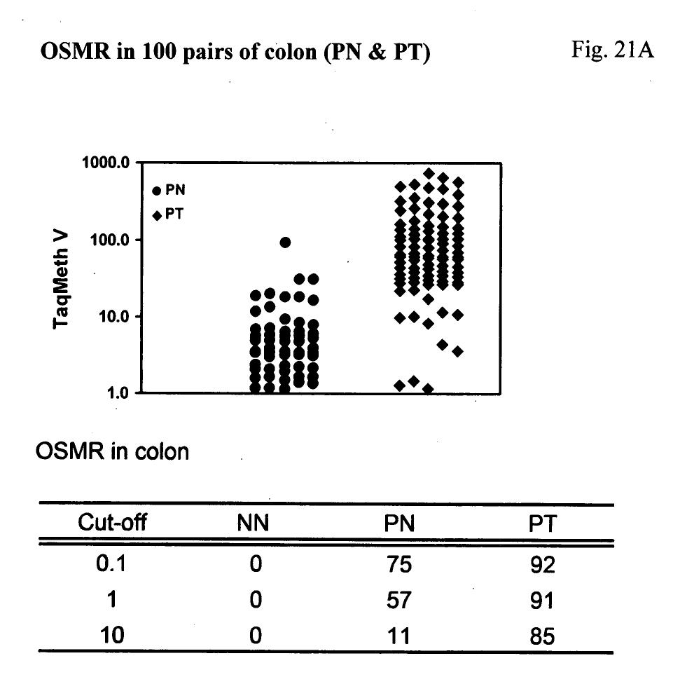

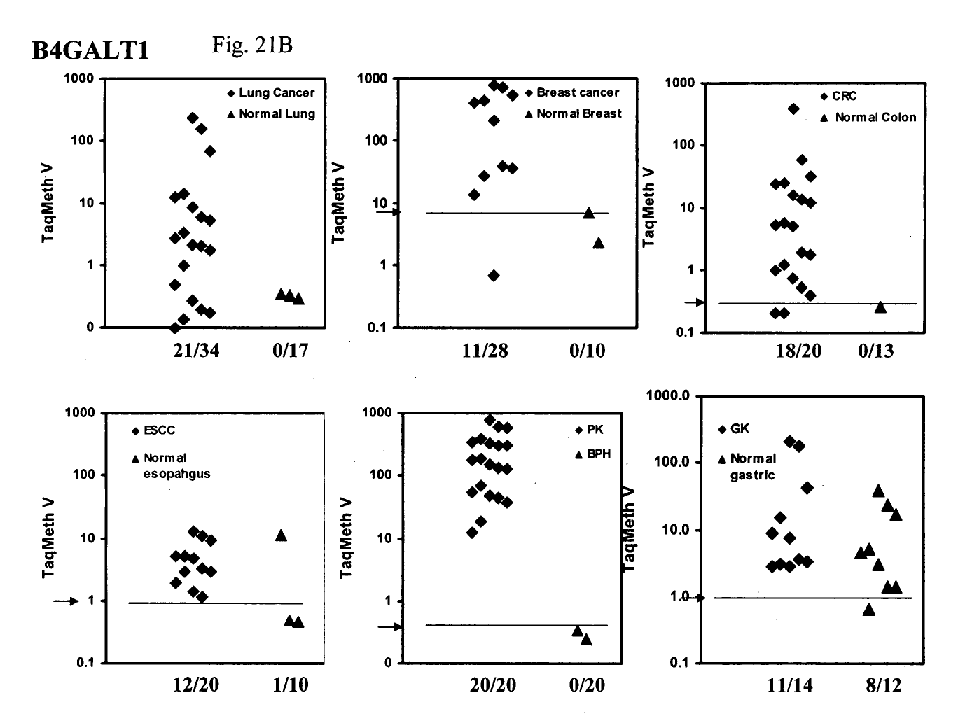

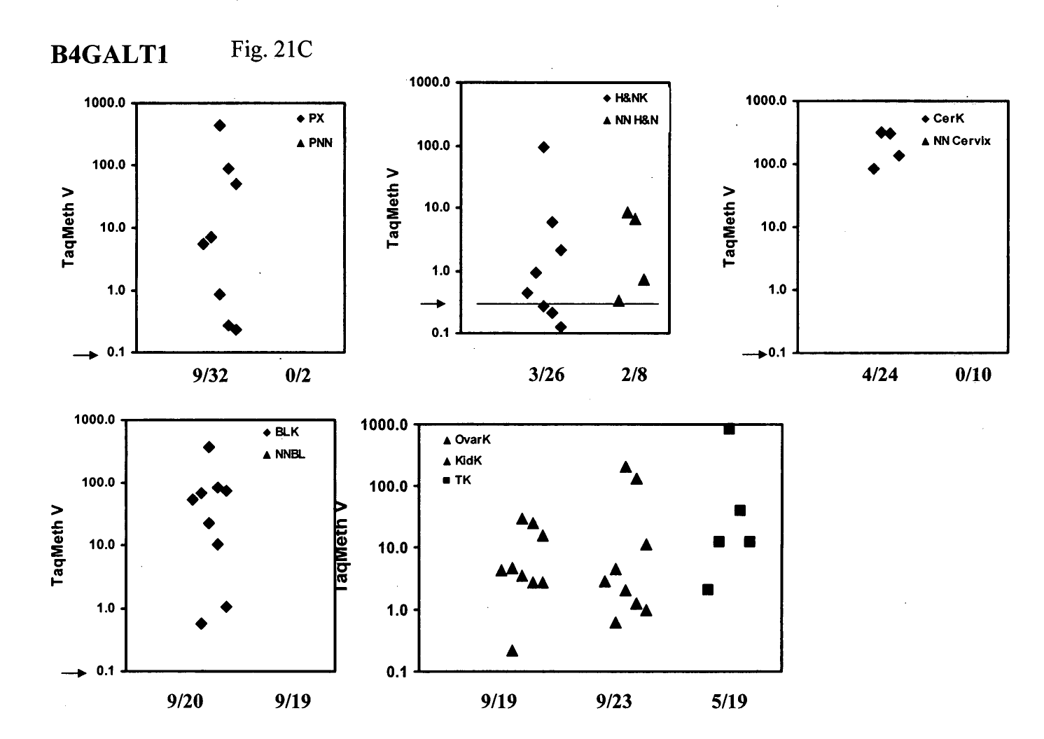

The methylation frequency of 8 potential methylation markers in different cancer types was investigated using primers given in Table 22. Results are summarized in Table 23 and FIG. 21A-21C. Methylation markers were retained based on absence of methylation in normal tissue (<10%) and a given sensitivity in tumors (>25%). This selection was complemented with markers passing the Fisher test (p<0.05).

Differential methylation was observed for:

PAK3 in lung, prostate, cervix and head and neck tumors;

NISCH in lung, gastric, kidney and head and neck tumors;

KIF1A in breast, pancreas, colon, cervix and head and neck tumors;

OGDHL in breast and colon tumors;

OSMR in colon and gastric tumors;

B4GALT1 in esophageal, lung, colon, prostate and bladder tumors;

MCAM in esophageal, prostate, thyroid and ovarian tumors;

SSBP2 in esophageal, colon, prostate, kidney and bladder tumors.

These data indicate that the above-listed markers can be used in any of the methods and kits disclosed in the specification for the Table 5 markers. Particularly, they can be used in any of the tumor types listed as demonstrating differential methylation.

### REFERENCES

The disclosure of each reference cited is expressly incorporated herein.

Reeves et al., U.S. Pat. No. 6,596,493

Sidransky, U.S. Pat. No. 6,025,127

Sidransky, U.S. Pat. No. 5,561,041

Nelson et al., U.S. Pat. No. 5,552,277

Herman, et al., U.S. Pat. No. 6,017,704

Baylin et al, U.S. Patent Application Publication No. 2003/0224040 A1

Belinsky et al., U.S. Patent Application Publication No. 2004/0038245 A1

Sidransky, U.S. Patent Application Publication No. 2003/0124600 A1

Sidransky, U.S. Patent Application Publication No. 2004/0081976 A1

Sukumar et al., U.S. Pat. No. 6,756,200 B2

Herman et al., U.S. Patent Application Publication No. 2002/0127572 A1

We claim:

1. A method for identifying a cell as neoplastic or predisposed to neoplasia, comprising:
detecting in a test cell epigenetic silencing of at least one gene listed in Table 5 wherein the test cell is selected from the group consisting of prostate, lung, breast, and colon cells;
identifying the test cell as neoplastic or predisposed to neoplasia.

2. The method of claim 1 wherein the cell is a prostate cell, and the at least one gene is selected from the group consisting of CD3D, APOC1, NBL1, ING4, LEF1, CENTD3, MGC15396, FKBP4, PLTP, TFAP2A, ATXN1, BMP2, ENPEP, MCAM, SSBP2, PDLIM3, PAK3, B4GALT1, and NDP.

3. The method of claim 1 wherein the cell is a prostate cell, and the at least one gene is selected from the group consisting of BMP2, ENPEP, MCAM, SSBP2, PAK3, B4GALT1, and NDP.

4. The method of claim 1 wherein the cell is a lung cell, and the at least one gene is selected from the group consisting of PHKA2, CBR3, CAMK4, HOXB5, ZNF198, RGS4, RBM15B, PDLIM3, PAK3, PIGH, TUBB4, B4GALT1, and NISCH.

5. The method of claim 1 wherein the cell is a lung cell, and the at least one gene is selected from the group consisting of PAK3, PIGH, TUBB4, B4GALT1, and NISCH.

6. The method of claim 1 wherein the cell is a breast cell, and the at least one gene is selected from the group consisting of BACH1, CKMT, GALE, HMG20B, KRT14, OGDHL, PON2, SESN1, KIF1A (kinesin family member 1A) PDLIM3 and MAL (T cell proliferation protein).

7. The method of claim 1 wherein the cell is a breast cell, and the at least one gene is selected from the group consisting of KIF1A (kinesin family member 1A) and MAL (T cell proliferation protein).

8. The method of claim 1 wherein the cell is a colon cell, and the at least one gene is selected from the group consisting of B4GALT1, C10orf119, C10orf13, CBR1, COPS4, COVA1, CSRP1, DARS, DNAJC10, FKBP14, FN3KRP, GANAB, HUS1, KLF11, MRPL4, MYLK, NELF, NETO2, PAPSS2, RBMS2, RHOB, SECTM1, SIRT2, SIRT7, SLC35D1, SLC9A3R1, TTRAP, TUBG2, FLJ20277, MYBL2, GPR116, OSMR, PC4, SLC39A4, UBE3A, PDLIM3, NRTK2, OGDHL, SFRP4, SSBP2, and UBE21.

9. The method of claim 1 wherein the cell is a colon cell, and the at least one gene is selected from the group consisting of GPR116, OSMR, PC4, SLC39A4, UBE3A, PDLIM3, NRTK2, OGDHL, SFRP4, SSBP2, and UBE21.

10. The method of claim 1 wherein epigenetic silencing of at least two genes is detected.

11. The method of claim 1 wherein epigenetic silencing is determined by measuring expression levels of at least one gene listed in Table 5.

12. The method of claim 1 wherein methylation of a CpG dinucleotide motif in the gene is detected.

13. The method of claim 12 wherein methylation is detected by contacting at least a portion of the gene with a methylation-sensitive restriction endonuclease, said endonuclease preferentially cleaving methylated recognition sites relative to non-methylated recognition sites, whereby cleavage of the portion of the gene indicates methylation of the portion of the gene.

14. The method of claim 12 wherein methylation is detected by contacting at least a portion of the gene with a methylation-sensitive restriction endonuclease, said endonuclease preferentially cleaving non-methylated recognition sites relative to methylated recognition sites, whereby cleavage of the portion of the gene indicates non-methylation of the portion of the gene provided that the gene comprises a recognition site for the methylation-sensitive restriction endonuclease.

15. The method of claim 12 wherein methylation is detected by:
contacting at least a portion of the gene of the test cell with a chemical reagent that selectively modifies a non-methylated cytosine residue relative to a methylated cytosine residue, or selectively modifies a methylated cytosine residue relative to a non-methylated cytosine residue; and
detecting a product generated due to said contacting.

16. The method of claim 15 wherein the step of detecting comprises amplification.

17. The method of claim 15 wherein the step of detecting comprises amplification with at least one primer that hybridizes to a sequence comprising a modified non-methylated CpG dinucleotide motif but not to a sequence comprising an unmodified methylated CpG dinucleotide motif thereby forming amplification products.

18. The method of claim 15 wherein the step of detecting comprises amplification with at least one primer that hybridizes to a sequence comprising an unmodified methylated CpG dinucleotide motif but not to a sequence comprising a modified non-methylated CpG dinucleotide motif thereby forming amplification products.

19. The method of claim 17 wherein the amplification products are detected using (a) a first oligonucleotide probe which hybridizes to a sequence comprising a modified non-methylated CpG dinucleotide motif but not to a sequence comprising an unmodified methylated CpG dinucleotide motif, (b) a second oligonucleotide probe that hybridizes to a sequence comprising an unmodified methylated CpG dinucleotide motif but not to sequence comprising a modified non-methylated CpG dinucleotide motif, or (c) both said first and second oligonucleotide probes.

20. The method of-claim 18 wherein the amplification products are detected using (a) a first oligonucleotide probe which hybridizes to a sequence comprising a modified non-methylated CpG dinucleotide motif but not to a sequence comprising an unmodified methylated CpG dinucleotide motif, (b) a second oligonucleotide probe that hybridizes to a sequence comprising an unmodified methylated CpG dinucleotide motif but not to sequence comprising a modified non-methylated CpG dinucleotide motif, or (c) both said first and second oligonucleotide probes.

21. The method of claim 15 wherein the product is detected by a method selected from the group consisting of hybridization, amplification, sequencing, electrophoresis, chromatography, and mass spectrometry

22. The method of claim 15 wherein the chemical reagent is hydrazine.

23. The method of claim 22 further comprising cleavage of the hydrazine-contacted at least a portion of the gene with piperidine.

24. The method of claim 15 wherein the chemical reagent comprises bisulfite ions.

25. The method of claim 24 further comprising treating the bisulfite ion-contacted at least a portion of the gene with alkali.

26. The method of claim 1 wherein the test cell is obtained from a surgical sample.

27. The method of claim 1 wherein the test cell is obtained from bone marrow, blood, serum, lymph, cerebrospinal fluid, saliva, sputum, stool, urine, or semen.

28. A method of reducing or inhibiting neoplastic growth of a prostate, lung, breast, or colon cell which exhibits epigenetic silenced transcription of at least one gene associated with a cancer, the method comprising:
restoring expression of a polypeptide encoded by the epigenetic silenced gene in the cell by contacting the cell with a CpG dinucleotide demethylating agent, wherein the gene is selected from those listed in Table 5, thereby reducing or inhibiting unregulated growth of the cell, with the proviso that if the cell is a breast or lung cell, the gene is not APC; and
testing expression of the gene in the cell to monitor response to the demethylating agent.

29. The method of claim 28 wherein the cell is a prostate cell, and the gene is selected from the group consisting of BMP2, ENPEP, MCAM, SSBP2, PAK3, B4GALT1, and NDP.

30. The method of claim 28 wherein the cell is a lung cell, and the gene is selected from the group consisting of PAK3, PIGH, TUBB4, B4GALT1, and NISCH.

31. The method of claim 28 wherein the cell is a breast cell, and the gene is selected from the group consisting of KIF1A (kinesin family member 1A) and MAL (T cell proliferation protein).

32. The method of claim 28 wherein the cell is a colon cell, and the gene is selected from the group consisting of GPR116, OSMR, PC4, SLC39A4, UBE3A, PDLIM3, NRTK2, OGDHL, SFRP4, SSBP2, and UBE21.

33. The method of claim 28 wherein the contacting is performed in vitro.

34. The method of claim 28 wherein the contacting is performed in vivo by administering the agent to a mammalian subject comprising the cell.

35. The method of claim 28 wherein the demethylating agent is selected from the group consisting of 5-aza-2′-deoxycytidine, 5-aza-cytidine, Zebularine, procaine, and L-ethionine.

36. A method of reducing or inhibiting neoplastic growth of a prostate, lung, breast, or colon cell which exhibits epigenetic silenced transcription of at least one gene associated with a cancer, the method comprising:
introducing a polynucleotide encoding a polypeptide into the cell which exhibits epigenetic silenced transcription of at least one gene listed in Table 5, wherein the polypeptide is encoded by said gene, wherein the polypeptide is expressed in the cell thereby restoring expression of the polypeptide in the cell, with the proviso that if the cell is a breast or lung cell, the gene is not APC.

37. The method of claim 36 wherein the cell is a prostate cell, a lung cell , a breast cell or a colon cell and the gene is selected from the group consisting of BMP2, ENPEP, MCAM, SSBP2, NDP, PAK3, B4GALT1, PIGH, TUBB4, NISCH, KIF1A (kinesin family member 1A), MAL (T cell proliferation protein), GPR116, OSMR, PC4, SLC39A4, UBE3A, PDLIM3, NRTK2, OGDHL, SFRP4, SSBP2, and UBE21.

38. A method of treating a prostate, lung, breast, or colon cancer patient, the method comprising:
administering a demethylating agent to the patient in sufficient amounts to restore expression of a tumor-associated methylation silenced gene selected from those listed in Table 5 in the patient's tumor, with the proviso that if the cell is a breast or lung cell, the gene is not APC; and
testing expression of the gene in cancer cells of the patient to monitor response to the demethylating agent.

39. The method of claim 38 wherein the cell is a prostate cell, a lung cell , a breast cell or a colon cell and the gene is selected from the group consisting of BMP2, ENPEP, MCAM, SSBP2, NDP, PAK3, B4GALT1, PIGH, TUBB4, NISCH. KIF1A (kinesin family member 1A), MAL (T cell proliferation protein), GPR116, OSMR, PC4, SLC39A4, UBE3A, PDLIM3, NRTK2, OGDHL, SFRP4, SSBP2, and UBE21.

40. A method of treating a prostate, lung, breast, or colon cancer patient, the method comprising:
administering to the patient a polynucleotide encoding a polypeptide, wherein the polypeptide is encoded by a gene listed in Table 5, wherein the polypeptide is expressed in the patient's tumor thereby restoring expression of the polypeptide in the tumor, with the proviso that if the cell is a breast or lung cell, the gene is not APC.

41. The method of claim 40 wherein the cell is a prostate cell, a lung cell , a breast cell or a colon cell and the gene is selected from the group consisting of BMP2, ENPEP, MCAM, SSBP2, NDP, PAK3, B4GALT1, NRTK2, OGDHL, SFRP4, SSBP2, PIGH, TUBB4, NISCH. KIF1A (kinesin family member 1A), MAL (T cell proliferation protein), GPR116, OSMR, PC4, SLC39A4, UBE3A, PDLIM3 and UBE21.

42. A method for selecting a therapeutic strategy for treating a prostate, lung, breast, or colon cancer patient, comprising:
identifying a gene selected from those listed in Table 5 whose expression in cancer cells of the patient is reactivated by a demethylating agent;
selecting a therapeutic agent which reactivates expression of the gene for treating said cancer patient, with the proviso that if the cancer cells are breast or lung cells, the gene is not APC.

43. The method of claim 42 wherein the therapeutic agent comprises a polynucleotide encoding the gene.

44. The method of claim 42 wherein the demethylating agent is 5-aza-2′-deoxycytidine.

45. The method of claim 42 wherein the therapeutic agent is 5-aza-2′-deoxycytidine.

46. The method of claim 42 wherein the cancer cells are selected from the group of cells consisting of lung, breast, colon, and prostate cells.

47. The method of claim 42 wherein the cancer cells are obtained from a surgical sample.

48. The method of claim 42 wherein the cancer cells are obtained from bone marrow, blood, serum, lymph, cerebrospinal fluid, saliva, sputum, stool, urine, or semen.

49. A kit for assessing methylation in a cell sample, comprising in a package:
a reagent that (a) modifies methylated cytosine residues but not non-methylated cytosine residues, or that (b); modifies non-methylated cytosine residues but not methylated cytosine residues; and
a pair of oligonucleotide primers that specifically hybridizes under amplification conditions to a region of a gene selected from those listed in Table 5, wherein the region is within about 1 kb of said gene's transcription start site.

50. The kit of claim 49 wherein the cell is a prostate cell, a lung cell, a breast cell or a colon cell and the gene is selected from the group consisting of BMP2, ENPEP, MCAM, SSBP2, NDP, PAK3, B4GALT1, PIGH, TUBB4, NISCH. KIF1A (kinesin family member 1A), MAL (T cell proliferation protein), GPR116, OSMR, PC4, SLC39A4, UBE3A, PDLIM3, NRTK2, OGDHL, SFRP4, SSBP2, and UBE21.

51. The kit of claim 49 wherein at least one of said pair of oligonucleotide primers hybridizes to a sequence comprising a modified non-methylated CpG dinucleotide motif but not to a sequence comprising an unmodified methylated CpG dinucleotide motif or wherein at least one of said pair of oligonucleotide primers hybridizes to a sequence comprising an unmodified methylated CpG dinucleotide motif but not to sequence comprising a modified non-methylated CpG dinucleotide motif.

52. The kit of claim 49 further comprising (a) a first oligonucleotide probe which hybridizes to a sequence comprising a modified non-methylated CpG dinucleotide motif but not to a sequence comprising an unmodified methylated CpG dinucleotide motif, (b) a second oligonucleotide probe that hybridizes to a sequence comprising an unmodified methylated CpG dinucleotide motif but not to sequence comprising a modified non-methylated CpG dinucleotide motif, or (c) both said first and second oligonucleotide probes.

53. The kit of claim 51 further comprising (a) a first oligonucleotide probe which hybridizes to a sequence comprising a modified non-methylated CpG dinucleotide motif but not to a sequence comprising an unmodified methylated CpG dinucleotide motif, (b) a second oligonucleotide probe that hybridizes to a sequence comprising an unmodified methylated CpG dinucleotide motif but not to sequence comprising a modified non-methylated CpG dinucleotide motif, or (c) both said first and second oligonucleotide probes.

54. The kit of claim 49 further comprising an oligonucleotide probe.

55. The kit of claim 49 further comprising a DNA polymerase for amplifying DNA.

56. A method to test compounds for their potential to treat cancer, comprising:
contacting the compound with a cancer cell selected from the group consisting of prostate, lung, breast, and colon cancer;
determining if expression of a gene selected from those listed in Table 5 is increased by the compound in the cell or if methylation of the gene is decreased by the compound in the cell.

57. The method of claim 56 wherein the cell is a prostate cell, a lung cell, a breast cell or a colon cell and the gene is selected from the group consisting of BMP2, ENPEP, MCAM, SSBP2, NDP, PAK3, B4GALT1,PIGH, TUBB4, NISCH. KIF1A (kinesin family member 1A), MAL (T cell proliferation protein), GPR116, OSMR, PC4, SLC39A4, UBE3A, PDLIM3, NRTK2, OGDHL, SFRP4, SSBP2, and UBE21.

58. A method to determine a prostate, lung, breast, or colon cancer patient's response to a chemotherapeutic agent, comprising:
treating the patient with the agent;
determining if expression of a gene selected from those listed in Table 5 is increased by the compound in cancer cells or if methylation of the gene is decreased by the compound in cancer cells.

59. The method of claim 58 wherein the cell is a prostate cell, a lung cell , a breast cell or a colon cell and the gene is selected from the group consisting of BMP2, ENPEP, MCAM, SSBP2, NDP, PAK3, B4GALT1,PIGH, TUBB4, NISCH, KIF1A (kinesin family member 1A), MAL (T cell proliferation protein), GPR116, OSMR, PC4, SLC39A4, UBE3A, PDLIM3, NRTK2, OGDHL, SFRP4, SSBP2, and UBE21.

60. A method of predicting a clinical response to treatment with doxorubicin of a subject in need thereof, comprising:
determining the state of methylation of a nucleic acid encoding CBR1 isolated from the subject,
wherein the state of methylation of the nucleic acid as compared with the state of methylation of the nucleic acid from a subject not in need of treatment is indicative of the level of CBR1; and
wherein CBR1 activates the anti-cancer activity of doxorubicin;
thereby predicting the clinical response to treatment of with doxorubicin.

61. A method of treating a cell proliferative disorder in a subject with an doxorubicin, comprising:
predicting a clinical response to treatment by determining the state of methylation of a nucleic acid isolated from the subject,
wherein the nucleic acid encodes CBR1 which activates the anti-cancer activity of doxorubicin; and
wherein the state of methylation of the nucleic acid as compared with the state of methylation of the nucleic acid from a subject not in need of treatment is indicative of the level of the CBR1.

62. The method of claim 60 or 61 further comprising the step of:
administering doxorubicin to the subject if a positive clinical response is predicted.

63. The method of claim 60 or 61 further comprising the step of:
administering the doxorubicin to the subject if the state of methylation of the nucleic acid is lower in the subject than in a subject not in need of treatment.
